(19) United States
(12) Patent Application Publication
(10) Pub. No.: US 2011/0152292 A1
(43) Pub. Date: Jun. 23, 2011

(54) SALTS COMPRISING A PYRIMIDINECARBOXYLIC ACID DERIVATIVE FOR COSMETIC USE

(75) Inventors: Michael Howard Rayner-Branes, Alsbach (DE); Thomas Rudolph, Darmstadt (DE); William-Robert Pitner, Frankfurt (DE); Jens Eichhorn, Reinheim (DE)

(73) Assignee: MERCK PATENT GESELLSCHAFT, Darmstadt (DE)

(21) Appl. No.: 13/002,182

(22) PCT Filed: Jun. 19, 2009
(86) PCT No.: PCT/EP2009/004447

§ 371 Date: Dec. 30, 2010

(30) Foreign Application Priority Data
Jul. 3, 2008 (DE) 10 2008 031 480.3

(51) Int. Cl. A61K 31/505; C07D 239/06; C07D 401/02; A61K 31/506; A61K 8/49; A61Q 19/08; A61Q 19/06; A61P 39/06; A61P 35/00; A61P 29/00; A61P 17/10; A61P 17/16

(52) U.S. Cl. 514/256; 544/335; 544/333

(57) ABSTRACT

The invention relates to novel compounds which comprise, as cationic or as anionic component, a pyrimidinecarboxylic acid derivative, in particular a derivative of ectoin or hydroxyectoin, to a process for the preparation thereof, and to the use thereof as ionic liquid or to the use thereof in pharmaceutical, cosmetic and dermatological formulations.

The invention relates to novel compounds which comprise, as cationic or as anionic component, a pyrimidinecarboxylic acid derivative, in particular a derivative of ectoin or hydroxyectoin, to a process for the preparation thereof, and to the use thereof as ionic liquid or to the use thereof in pharmaceutical, cosmetic and dermatological formulations.

Ionic liquids or liquid salts are ionic species which consist of an organic cation and a generally inorganic anion. They do not contain any neutral molecules, and generally have melting points below 373 K. A multiplicity of compounds which are used as ionic liquids are known from the prior art.

In particular, they are also the subject-matter of a series of patents and patent applications. Thus, solvent-free ionic liquids were disclosed for the first time by Hurley and Wier in a series of US patents (U.S. Pat. No. 2,446,331, U.S. Pat. No. 2,446,339 and U.S. Pat. No. 2,446,350). These “salts which are molten at room temperature” comprise AlCl₃ and a multiplicity of n-alkylpyridinium halides.

In recent years, some review articles have been published on this topic (R. Sheldon “Catalytic reactions in ionic liquids”, Chem. Commun., 2001, 2399-2407; M. J. Earle, K. R. Seddon “Ionic liquids. Green solvent for the future”, Pure Appl. Chem., 72 (2000), 1391-1398; P. Wasserscheid, W. Keim “Ionische Flüssigkeiten—neue Lösungen für die Übergangsmetallkatalyse” [Ionic Liquids—Novel Solutions for Transition-Metal Catalysis], Angew. Chem., 112 (2000), 3926-3945; T. Welton “Room temperature ionic liquids. Solvents for synthesis and catalysis”, Chem. Rev., 92 (1999), 2071-2083; R. Hagiwara, Ya. Ito “Room temperature ionic liquids of alkylimidazolium cations and fluoroanions”, Journal of Fluorine Chem., 105 (2000), 221-227).

The properties of ionic liquids, such as, for example, the melting point, the thermal and electrochemical stability and the viscosity, are strongly influenced by the nature of the anion and cation. The polarity and hydrophilicity or lipophilicity can be adjusted through the choice of a suitable cation/anion pair. Each new anion and each new cation opens up further possibilities for tuning the properties of ionic liquids. There is therefore a basic demand for novel ionic liquids having varied properties which facilitate additional possibilities with respect to their use.

Ectoin ((S)-1,4,5,6-tetrahydro-2-methyl-4-pyrimidinecarboxylic acid) and its derivative hydroxyectoin ((S,S)-1,4,5,6-tetrahydro-5-hydroxy-2-methyl-4-pyrimidinecarboxylic acid) are naturally occurring amino acids which are involved in the osmoregulation of plants and microorganisms and which can be isolated from these organisms. Ectoin and hydroxyectoin are used as active compounds in skin-care and skin-protecting compositions, where they act as stabiliser for proteins and cell structures and against external stress factors, such as, for example, UV irradiation and dryness.

The object of the present invention is therefore to provide novel compounds which, besides the classical areas of application of ionic liquids, also open up new possible uses in the area of medicaments or cosmetics.

This object is achieved in accordance with the invention by the characterising features of the main claim and the co-ordinate claims.

Surprisingly, it has now been found that it is possible to prepare derivatives of ectoin or hydroxyectoin which are ionic liquids and whose properties with respect to solubility and bioavailability can be modified through their counterion.

The present invention therefore relates to a compound comprising a cationic component and an anionic component, in which a pyrimidinecarboxylic acid derivative represents the cationic component or the anionic component, where ectoin hydrochloride is excluded.

The salts according to the invention are used here in the same areas which are also already known for ectoin and its derivative hydroxyectoin.

The compound according to the invention preferably comprises, as cationic component, a pyrimidinecarboxylic acid derivative which has formed through protonation of a neutral pyrimidinecarboxylic acid derivative, or, as anionic component, a pyrimidinecarboxylic acid derivative which has formed through deprotonation of the neutral pyrimidinecarboxylic acid derivative. The protonation here takes place on the nitrogen atom adjacent to the carboxyl group, whereas in the case of deprotonation, the carboxyl group is converted into its carboxylate.

The compounds according to the invention particularly preferably comprise derivatives of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid).

The compounds according to the invention are preferably ionic liquids and/or cosmetic active compounds.

The compounds according to the invention advantageously exhibit very good flexibility with respect to their solubility and their bioavailability, making them highly suitable as active compounds, in particular in dermatological formulations or skin-care products. The properties of solubility and bioavailability can be varied very simply here via the counterion of the pyrimidinecarboxylic acid ion. The choice of the corresponding counterion presents the person skilled in the art with absolutely no difficulties.

Compounds according to the invention having the typical anions for ionic liquids, such as, for example, imides, triflates, fluoroalkylphosphates, are preferably employed as catalytic materials in the sense of ionic liquids. The cosmetically active compounds used are preferably lipophilic ionic combinations of the salts according to the invention. These allow the ectoin derivatives according to the invention to be transported into the oil phase, causing, in particular, a synergistic effect with the ectoin in the water phase to occur in the case of selected combinations.

With respect to the choice of counterion of the compound in accordance with the present invention, there are no restrictions per se. If the pyrimidinecarboxylic acid derivative is in the form of its anion, the associated cations are preferably organic cations, particularly preferably ammonium, phosphonium, uronium, thiouronium or guanidinium cations, or heterocyclic cations. If the pyrimidinecarboxylic acid derivative is in the form of its cation, the associated anion is preferably an anion which is typical for ionic liquids.

The compounds which are preferred in accordance with the invention can be described, for example, by the general formula (I)

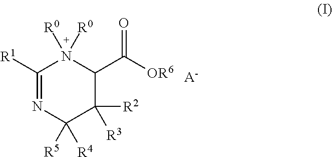

in which the radicals are defined as follows:
R⁰=H or alkyl having 1-12 C atoms,
R¹=H or alkyl having 1-4 C atoms,
R², R³, R⁴, R⁵=each, independently of one another,
   H, OH, NH₂ or alkyl having 1-4 C atoms,
R⁶=H or alkyl having 1-8 C atoms,
A⁻=[R⁹C(O)O]⁻, [R^{F}C(O)O]⁻, [R⁹SO₃]⁻, [R^{F}SO₃]⁻, [R⁹OSO₃]⁻, [R^{F}OSO₃]⁻, [(R^{F}SO₂)₂N]⁻, [(R⁹SO₂)₂N]⁻, [(R^{F}C(O))₂N]⁻, [(R⁹C(O))₂N]⁻, [(R^{F}SO₂)(R^{F}C(O))N]⁻, [(R⁹SO₂)(R⁹C(O))N]⁻, [(FSO₂)₃C]⁻, [(R^{F}SO₂)₃C]⁻, [(R⁹SO₂)₃C]⁻, [CCl₃C(O)O]⁻, [(CN)₃C]⁻, [(CN)₂CR⁹]⁻, [(R⁹O(O)C)₂CR⁹]⁻, [P(R^{F})_{y}F_{6-y}]⁻, [P(C₆F₅)_{y}F_{6-y}]⁻, [R⁹₂P(O)O]⁻, [R⁹P(O)O₂]²⁻, [(R⁹O)₂P(O)O]⁻, [(R⁹O)P(O)O₂]²⁻, [(R⁹O)(R⁹)P(O)O]⁻, [R^{F}₂P(O)O]⁻, [R^{F}P(O)O₂]²⁻, [(R^{F})₂P(O)]₂N⁻, [BF_{Z}R^{F}_{4-z}]⁻, [BF_{Z}(CN)_{4-z}]⁻, [B(C₆H₅)₄]⁻, [B(C₆F₅)₄]⁻, [B(OR⁹)₄]⁻, [N(CF₃)₂]⁻, [N(CN)₂]⁻, [AlCl₄]⁻, [SiF₆]²⁻, [R⁹⁰SO₃]⁻, [HSO₄]⁻, Br⁻, [SO₄]²⁻, [SCN]⁻, [NO₃]⁻, [AlCl₄]⁻, [Al₂Cl₇]⁻, [SnCl₃]⁻, [CO₃]²⁻, [SbF₆]⁻ and [AsF₆]⁻,
   where the substituents R^{F} each, independently of one another, denote
   perfluorinated and straight-chain or branched alkyl having 1-20 C atoms,
   perfluorinated and straight-chain or branched alkenyl having 2-20 C atoms and one or more double bonds,
   perfluorinated and saturated, partially or fully unsaturated cycloalkyl having 3-7 C atoms, in particular phenyl, which may be substituted by perfluoroalkyl groups,
   where the substituents R^{F} may be bonded to one another in pairs by a single or double bond,
   and where one or two carbon atoms of the R^{F} which are not adjacent and are in the α-position to the heteroatom may be replaced by atoms and/or atom groups selected from the group —O—, —C(O)—, —S—, —S(O)—, —SO₂—, —SO₂O—, —N═, —N═N—, —NH—, —NR′—, —PR′— and —P(O)R′— or may have an end group R′—O—SO₂— or R′—O—C(O)—, where R′ denotes unfluorinated, partially fluorinated or perfluorinated alkyl having 1-6 C atoms, saturated or partially unsaturated cycloalkyl having 3-7 C atoms, unsubstituted or substituted phenyl, including —C₆F₅, or an unsubstituted or substituted heterocycle,
   where the substituents R⁹ each, independently of one another, denote
      H,
      straight-chain or branched alkyl having 1-20 C atoms,
      straight-chain or branched alkenyl having 2-20 C atoms and one or more double bonds,
      saturated, partially or fully unsaturated cycloalkyl having 3-7 C atoms, in particular phenyl, which may be substituted by alkyl groups,
   where a plurality of substituents R⁹ may be bonded to one another in pairs by a single or double bond,
   and where one or two carbon atoms of the R⁹ which are not adjacent and are not in the α-position to the heteroatom may be replaced by atoms and/or atom groups selected from the group —O—, —C(O)—, —S—, —S(O)—, —SO₂—, —SO₂O—, —N═, —N═N—, —NH—, —NR′—, —PR′—, —P(O)R′—, —P(O)R′O—, —OP(O)R′O—, —PR′₂═N—, —C(O)NH—, —C(O)NR′—, —SO₂NH— and —SO₂NR′, where R′ denotes unfluorinated, partially fluorinated or perfluorinated alkyl having 1-6 C atoms, saturated or partially unsaturated cycloalkyl having 3-7 C atoms, unsubstituted or substituted phenyl, including —C₆F₅, or an unsubstituted or substituted heterocycle,
   and where
   y=0, 1, 2, 3, 4, 5 or 6 and
   z=0, 1, 2, 3 or 4.

Alternatively, the compounds which are preferred in accordance with the invention can be described, for example, by the general formula (II)

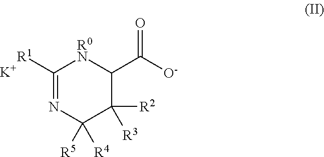

in which the radicals are defined as follows:
R⁰=H or alkyl having 1-12 C atoms,
R¹=H or alkyl having 1-4 C atoms,
R², R³, R⁴, R⁵=each, independently of one another,
   H, OH, NH₂ or alkyl having 1-4 C atoms,
K⁺=ammonium [N(R⁷)₄]⁺,
   phosphonium [N(R⁷)₄]⁺,
   uronium [((R⁷)₂N)—C(═OR⁸)(N(R⁷)₂)]⁺,
   thiouronium [((R⁷)₂N)—C(═SR⁸)(N(R⁷)₂)]⁺,
   guanidinium [C((N(R⁷)₂)₃]⁺,
   sulfonium [S(R⁷)₃]⁺
   or a heterocyclic cation [HetN]⁺,
   where R⁷, R⁸ each, independently of one another, denote
      —H, with the proviso that, in the case of [(R⁷)₄N]⁺,
      a maximum of two R⁷ are H and that H is excluded for R⁸,
      OR′, NR′₂, with the proviso that,
      in the case of [(R⁷)₄N]⁺, a maximum of one R⁷ is OR′, NR′₂ and that OR′, NR′₂ are excluded in the case of [((R⁷)₂N)—C(═OR⁸)(N(R⁷)₂)]⁺ and R(R⁷)₂N)—C(═SR⁸)(N(R⁷)₂)⁺,
      CN, with the proviso that
      CN is excluded in the case of [N(R⁷)₄]⁺, [P(R⁷)₄]⁺, R(R⁷)₂N)—C(═OR⁸)(N(R⁷)₂)⁺ and [((R⁷)₂N)—C(═SR⁸)(N(R⁷)₂)]⁺,
      straight-chain or branched alkyl having 1-20 C atoms,
      straight-chain or branched alkenyl having 2-20 C atoms and one or more double bonds,
      straight-chain or branched alkynyl having 2-20 C atoms and one or more triple bonds,
      saturated, partially or fully unsaturated cycloalkyl having 3-7 C atoms, which may be substituted by alkyl groups having 1-6 C atoms,
   where one or more R⁷, R⁸ may be partially or fully substituted by halogens, in particular —F and/or —Cl, or partially by —OH, —OR′, —CN, —C(O)OH, —C(O)NR′₂, —SO₂NR′₂, —C(O)X, —SO₂OH, —SO₂X, —NO₂ or —(CH₂)ₙ-phenyl, and where one or two carbon atoms of the R⁷ which are not adjacent and are not in the α-position may be replaced by atoms and/or atom groups selected from the group —O—, —S—, —S(O)—, —SO₂—, —SO₂O—, —C(O)—, —C(O)O—, —N⁺R′₂—, —P(O)R′O—, —C(O)NR′—, —SO₂NR′—, —OP(O)R′O—, —P(O)(NR′₂)NR′—, —PR′₂═N— or —P(O)R′—,
   where n=1-4, R′=H, unfluorinated, partially fluorinated or perfluorinated C₁- to C₆-alkyl, C₃- to C₇-cycloalkyl, unsubstituted or substituted phenyl and X=halogen,
   and where the heterocyclic cation [HetN]⁺ is selected from the group

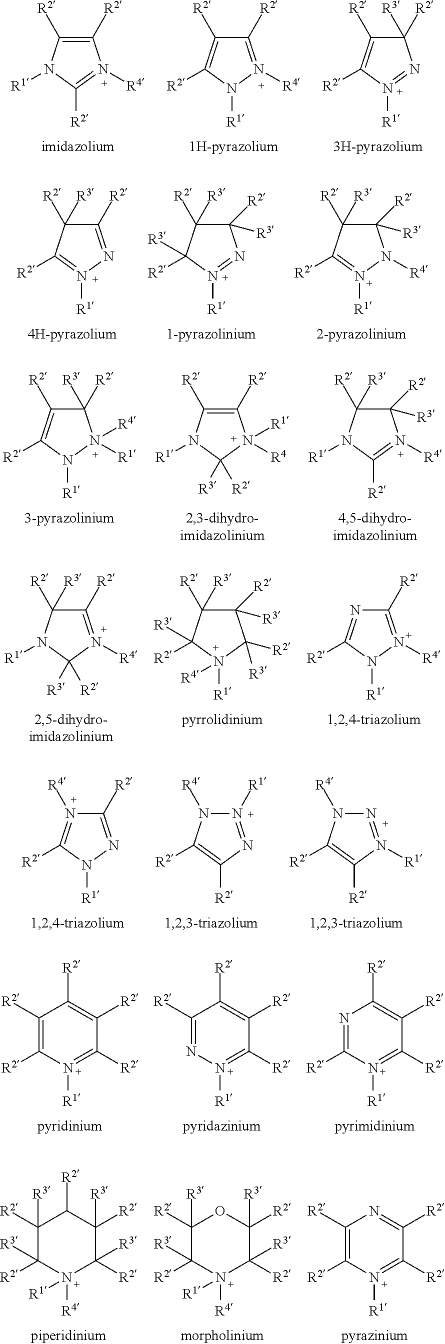

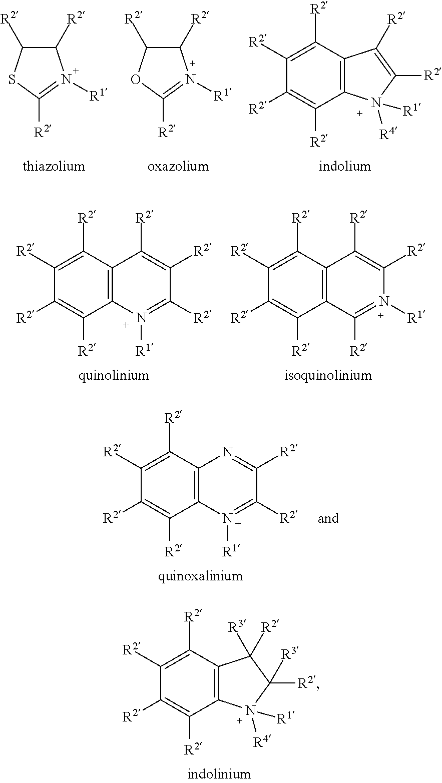

where the substituents R¹′, R²′, R³′ and R⁴′ each, independently of one another, denote
   H, —CN, —OR′, —NR′₂, —P(O)R′₂, —P(O)(OR′)₂, —P(O)(NR′₂)₂, —C(O)R′, —C(O)OR′,
   straight-chain or branched alkyl having 1-20 C atoms,
   straight-chain or branched alkenyl having 2-20 C atoms and one or more double bonds,
   straight-chain or branched alkynyl having 2-20 C atoms and one or more triple bonds,
   saturated, partially or fully unsaturated cycloalkyl having 3-7 C atoms, which may be substituted by alkyl groups having 1-6 C atoms,
   saturated, partially or fully unsaturated heteroaryl,
   heteroaryl-C₁-C₆-alkyl or aryl-C₁-C₆-alkyl,
where the substituents R¹′, R²′, R³′ and/or R⁴′ together may also form a ring system,
where one or more substituents R¹′ to R⁴′ may be partially or fully substituted by halogens, in particular —F and/or —Cl, or —OH, —OR′, —CN, —C(O)OH, —C(O)NR′₂, —SO₂NR′₂, —C(O)X, —SO₂OH, —SO₂X, —NO₂ or —(CH₂)ₙ-phenyl, but where R¹′ and R⁴′ cannot simultaneously be fully substituted by halogens,
where one or two substituent R¹′ to R⁴′ carbon atoms which are not adjacent and are not bonded to the heteroatom may be replaced by atoms and/or atom groups selected from —O—, —S—, —S(O)—, —SO₂—, —SO₂O—, —C(O)—, —C(O)O—, —N⁺R′₂—, —P(O)R′O—, —C(O)NR′—, —OP(O)R′O—, —P(O)(NR′₂)NR′—, —PR′₂═N— or —P(O)R′—,
and where n=1-4, R′=H, unfluorinated, partially fluorinated or perfluorinated C₁- to C₆-alkyl, C₃- to C₇-cycloalkyl, unsubstituted or substituted phenyl and X=halogen.

Fully unsaturated substituents in the sense of the present invention are also taken to mean aromatic substituents.

In accordance with the invention, suitable substituents R⁷ and R⁸ of a compound of the formula (II) are preferably, besides H: C₁— to C₂₀-, in particular C₁- to C₁₄-alkyl groups, and saturated or unsaturated, i.e. also aromatic, C₃- to C₇-cycloalkyl groups, which may be substituted by C₁- to C₆-alkyl groups, in particular phenyl.

The substituents R⁷ in a compound of the formula (II) may be identical or different. The substituents R⁷ are preferably different. In the case of ammonium, it is particularly preferred either for in each case two of the four substituents R⁷ to be identical or for three to be identical and one to be different. In the case of sulfonium, it is particularly preferred for two of the three substituents R⁷ to be identical.

Particularly preferred substituents R⁷ of the ammonium and phosphonium ion in a compound of the formula (II) are, independently of one another, methyl, ethyl, isopropyl, propyl, butyl, sec-butyl, tert-butyl, pentyl, hexyl, octyl, decyl or tetradecyl.

Up to four substituents of the guanidinium cation [C(N(R⁷)₂)₃]⁺ may also be bonded in pairs in such a way that mono-, bi- or polycyclic cations arise. Without restricting generality, examples of such guanidinium cations are:

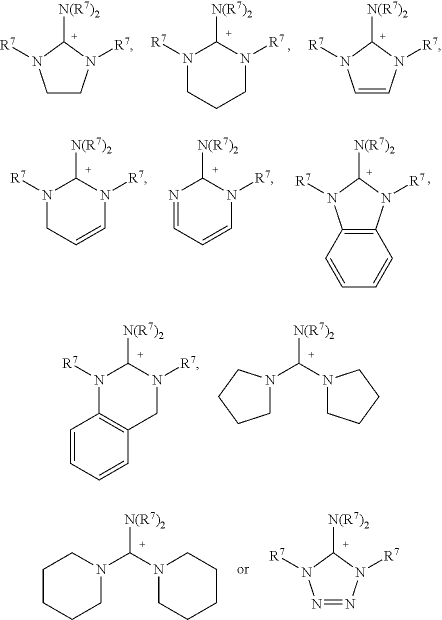

where the substituents R⁷ each, independently of one another, have a meaning indicated above or a particularly preferred meaning.

Up to four substituents of the uronium cation [((R⁷)₂N)—C(═OR⁸)(N(R⁷)₂)]⁺ or thiouronium cation [((R⁷)₂N)—C(═SR⁸)(N(R⁷)₂)]⁺ may also be bonded in pairs in such a way that mono-, bi- or polycyclic cations arise. Without restricting generality, examples of such cations are indicated below, where Y═O or S:

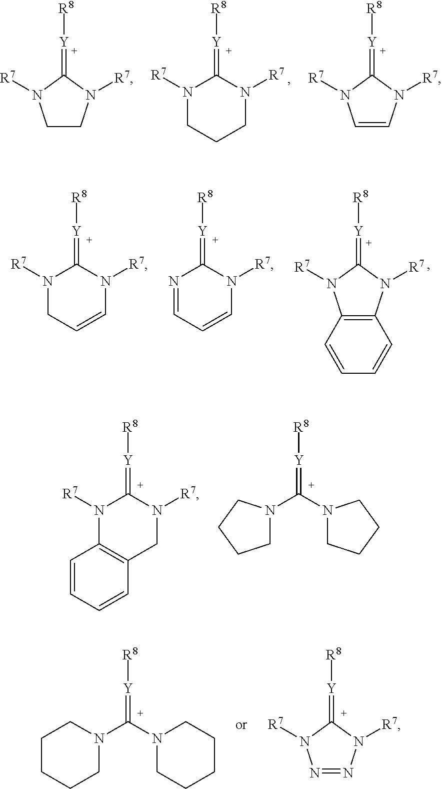

where the substituents R⁷ and R⁸ may each, independently of one another, have a meaning indicated above or a particularly preferred meaning.

The carbocycles or heterocycles of the guanidinium, uronium or thiouronium cations indicated above as particularly preferred examples may optionally also be substituted by C₁- to C₆-alkyl, C₁- to C₆-alkenyl, NO₂, F, Cl, Br, I, OH, C₁-C₆-alkoxy, SCF₃, SO₂CF₃, COON, SO₂NR′₂, SO₂X or SO₃H or substituted or unsubstituted phenyl or an unsubstituted or substituted heterocycle, where X and R′ have a meaning indicated above.

The substituents R⁷ and R⁸ of the guanidinium, uronium or thiouronium cation in a compound of the formula (II) are in each case, independently of one another, preferably a straight-chain or branched alkyl group having 1 to 10 C atoms. The substituents R⁷ and R⁸ here may be identical or different. R⁷ and R⁸ are particularly preferably each, independently of one another, methyl, ethyl, n-propyl, isopropyl, n-butyl, tert-butyl, sec-butyl, phenyl or cyclohexyl, very particularly preferably methyl, ethyl, n-propyl, isopropyl or n-butyl.

In accordance with the invention, suitable substituents R^{1′} to R^{4′} of the heterocyclic cation of a compound of the formula (II) are preferably, besides H: C₁— to C₂₀-, in particular C₁- to C₁₂-alkyl groups, and saturated or unsaturated, i.e. also aromatic, C₃- to C₇-cycloalkyl groups, which may be substituted by C₁- to C₆-alkyl groups, in particular phenyl.

The substituents R^{1′} and R^{4′} are each, independently of one another, particularly preferably methyl, ethyl, isopropyl, propyl, butyl, sec-butyl, tert-butyl, pentyl, hexyl, octyl, decyl, cyclohexyl, phenyl or benzyl. They are very particularly preferably methyl, ethyl, n-butyl or hexyl. In pyrrolidinium, piperidinium or indolinium compounds, the two substituents R^{1′} and R^{4′} are preferably different.

The substituent R^{2′} or R^{3′} is in each case, independently of one another, in particular H, methyl, ethyl, isopropyl, propyl, butyl, sec-butyl, tert-butyl, cyclohexyl, phenyl or benzyl. R^{2′} is particularly preferably H, methyl, ethyl, isopropyl, propyl, butyl or sec-butyl. R^{2′} and R^{3′} are very particularly preferably H.

Without restricting generality, the C₁-C₁₂-alkyl group is, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl or dodecyl.

A straight-chain or branched alkenyl having 2 to 20 C atoms, where a plurality of double bonds may also be present, is, for example, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉, preferably allyl, 2- or 3-butenyl, isobutenyl, sec-butenyl, furthermore preferably 4-pentenyl, isopentenyl or hexenyl.

A straight-chain or branched alkynyl having 2 to 20 C atoms, where a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, particularly preferably ethynyl, 1- or 2-propynyl, 2- or 3-butynyl, 4-pentynyl, 3-pentynyl or hexynyl.

Without restricting generality, aryl-C₁-C₆-alkyl denotes, for example, benzyl, phenylethyl, phenylpropyl, phenylbutyl, phenylpentyl or phenylhexyl, where both the phenyl ring and also the alkylene chain may, as described above, be partially or fully substituted by halogens, in particular —F and/or —Cl, or partially by —OH, —OR′, —CN, —C(O)OH, —C(O)NR′₂, —SO₂NR′₂, —C(O)X, —SO₂OH, —SO₂X, —NO₂ or —(CH₂)ₙ-phenyl, where n=1-4.

Unsubstituted saturated or partially or fully unsaturated cycloalkyl groups having 3-7 C atoms are therefore cyclopropyl, cyclobutyl, cyclopentyl, cyclohexyl, cycloheptyl, cyclopentenyl, cyclopenta-1,3-dienyl, cyclohexenyl, cyclohexa-1,3-dienyl, cyclohexa-1,4-dienyl, phenyl, cycloheptenyl, cyclohepta-1,3-dienyl, cyclohepta-1,4-dienyl or cyclohepta-1,5-dienyl, each of which may be substituted by C₁- to C₆-alkyl groups, where in turn the cycloalkyl group or the cycloalkyl group which is substituted by C₁- to C₆-alkyl groups may also be substituted by halogen atoms, such as F, Cl, Br or I, in particular F or Cl, or by —OH, —OR′, —ON, —C(O)OH, —C(O)NR′₂, —SO₂NR′₂, —C(O)X, —SO₂OH, —SO₂X, —NO₂ or —(CH₂)ₙ-phenyl, where n=1-4.

In the substituents R⁷, R⁸ and R^{1′} to R^{4′}, one or two carbon atoms which are not adjacent and are not bonded in the α-position to the heteroatom may also be replaced by atoms and/or atom groups selected from the group —O—, —S—, —S(O)—, —SO₂—, —SO₂O—, —C(O)—, —C(O)O—, —N⁺R′₂—, —P(O)R′O—, —C(O)NR′—, —SO₂NR′—, —OP(O)R′O—, —P(O)(NR′₂)NR′—, —PR′₂═N— or —P(O)R′—, where R′=unfluorinated, partially fluorinated or perfluorinated C₁- to C₆-alkyl, C₃- to C₇-cycloalkyl, unsubstituted or substituted phenyl.

Without restricting generality, examples of substituents R⁷, R⁸ and R^{1′} to R^{4′} modified in this way are:

—OCH₃, —OCH(CH₃)₂, —CH₂OCH₃, —CH₂—CH₂—O—CH₃, —C₂H₄OCH(CH₃)₂, —C₂H₄C₂H₅, —C₂H₄SCH(CH₃)₂, —S(O)CH₃, —SO₂CH₃, —SO₂C₆H₅, —SO₂C₃H₇, —SO₂CH(CH₃)₂, —SO₂CH₂CF₃, —CH₂SO₂CH₃, —O—C₄H₈—O—C₄H₉, —CF₃, —C₂F₅, —C₃F₇, —C₄F₉, —C(CF₃)₃, —CF₂SO₂CF₃, —C₂F₄N(C₂F₅)C₂F₅, —CHF₂, —CH₂CF₃, —C₂F₂H₃, —C₃H₆, —CH₂C₃F₇, —C(CFH₂)₃, —CH₂C(O)OH, —CH₂C₆H₅, —C(O)C₆H₅ and —P(O)(C₂H₅)₂.

In R′, C₃- to C₇-cycloalkyl is, for example, cyclopropyl, cyclobutyl, cyclopentyl, cyclohexyl or cycloheptyl.

In R′, substituted phenyl denotes phenyl which is substituted by C₁- to C₆-alkyl, C₁- to C₆-alkenyl, NO₂, F, Cl, Br, I, OH, C₁-C₆-alkoxy, SCF₃, SO₂CF₃, COON, SO₂X′, SO₂NR″₂ or SO₃H, where X′ denotes F, Cl or Br and R″ denotes unfluorinated, partially fluorinated or perfluorinated C₁- to C₆-alkyl or C₃- to C₇-cycloalkyl as defined for R′, for example o-, m- or p-methylphenyl, o-, m- or p-ethylphenyl, o-, m- or p-propylphenyl, o-, m- or p-isopropylphenyl, o-, m- or p-tert-butylphenyl, o-, m- or p-nitrophenyl, o-, m- or p-hydroxyphenyl, o-, m- or p-methoxyphenyl, o-, m- or p-ethoxyphenyl, o-, m-, p-(trifluoromethyl)phenyl, o-, m-, p-(trifluoromethoxy)phenyl, o-, m-, p-(trifluoromethylsulfonyl)phenyl, o-, m- or p-fluorophenyl, o-, m- or p-chlorophenyl, o-, m- or p-bromophenyl, o-, m- or p-iodophenyl, further preferably 2,3-, 2,4-, 2,5-, 2,6-, 3,4- or 3,5-dimethylphenyl, 2,3-, 2,4-, 2,5-, 2,6-, 3,4- or 3,5-dihydroxyphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

In R^{1′} to R^{4′}, heteroaryl denotes a saturated or unsaturated mono- or bicyclic heterocyclic radical having 5 to 13 ring members, where 1, 2 or 3 N and/or 1 or 2 S or O atoms may be present and the heterocyclic radical may be mono- or polysubstituted by C₁- to C₆-alkyl, C₁- to C₆-alkenyl, NO₂, F, Cl, Br, I, OH, C₁-C₆-alkoxy, SCF₃, SO₂CF₃, COOH, SO₂X′, SO₂NR″₂ or SO₃H, where X′ and R″ have a meaning indicated above.

The heterocyclic radical here is preferably substituted or unsubstituted 2- or 3-furyl, 2- or 3-thienyl, 1-, 2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -4- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8-cinnolinyl, 2-, 4-, 5-, 6-, 7- or 8-quinazolinyl or 1-, 2- or 3-pyrrolidinyl.

In accordance with the invention, heteroaryl-C₁-C₆-alkyl is taken to mean, analogously to aryl-C₁-C₆-alkyl, for example pyridinylmethyl, pyridinylethyl, pyridinylpropyl, pyridinylbutyl, pyridinylpentyl, pyridinylhexyl, where the heterocycles described above may furthermore be linked to the alkylene chain in this way.

The cations of the compound of the formula (II) according to the invention are preferably ammonium, phosphonium, guanidinium, sulfonium or heterocyclic cations.

The cations of the compound of the formula (II) according to the invention are particularly preferably ammonium ions [N(R⁷)₄]⁺ and heterocyclic cations [HetN]⁺, where R⁷ in each case, independently of one another, denotes
H, with the proviso that a maximum of two R⁷ are H,
OR′, NR′₂, with the proviso that a maximum of one R⁷ is OR′, NR′₂,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
   where one or more R⁷ may be partially or fully substituted by halogens, in particular —F and/or —Cl, or partially by —OH, —OR′, —CN, —C(O)OH, —C(O)NR′₂, —SO₂NR′₂, —C(O)X, —SO₂OH, —SO₂X, —NO₂ or —(CH₂)ₙ-phenyl, and where one or two carbon atoms of the R⁷ which are not adjacent and are not in the α-position may be replaced by atoms and/or atom groups selected from the group —O—, —S—, —S(O)—, —SO₂—, —SO₂O—, —C(O)—, —C(O)O—, —N⁺R′₂—, —P(O)R′O—, —C(O)NR′—, —SO₂NR′—, —OP(O)R′O—, —P(O)(NR′₂)NR′—, —PR′₂═N— or —P(O)R′—,
   where n=1-4, R′=H, unfluorinated, partially fluorinated or perfluorinated C₁- to C₆-alkyl, C₃- to C₇-cycloalkyl, unsubstituted or substituted phenyl and X=halogen.

The heterocyclic cation [HetN]⁺ is particularly preferably selected from the group

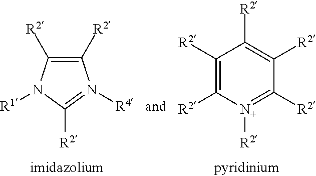

where the substituents R²′ denote H and
where the substituents R¹′ and R⁴′ each, independently of one another, denote
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
saturated, partially or fully unsaturated heteroaryl,
heteroaryl-C₁-C₆-alkyl or aryl-C₁-C₆-alkyl,
   where the substituents R¹′ and R⁴′ together may also form a ring system,
   where the substituents R¹′ and/or R⁴′ may be partially or fully substituted by halogens, in particular —F and/or —Cl, or —OH, —OR′, —CN, —C(O)OH, —C(O)NR′₂, —SO₂NR′₂, —C(O)X, —SO₂OH, —SO₂X, —NO₂ or —(CH₂)ₙ-phenyl, but where R¹′ and R⁴′ cannot simultaneously be fully substituted by halogens,
   where one or two substituent R¹′ to R⁴′ carbon atoms which are not adjacent and are not bonded to the heteroatom may be replaced by atoms and/or atom groups selected from the group —O—, —S—, —S(O)—, —SO₂—, —SO₂O—, —C(O)—, —C(O)O—, —P(O)R′O—, —C(O)NR′—, —SO₂NR′—, —OP(O)R′O—, —P(O)(NR′₂)NR′—, —PR′₂═N— or —P(O)R′—,
   and where n=1-4, R′=H, unfluorinated, partially fluorinated or perfluorinated C₁- to C₆-alkyl, C₃- to C₇-cycloalkyl, unsubstituted or substituted phenyl and X=halogen.

Very particularly preferred cations of the compound of the formula (II) according to the invention are selected from the group consisting of 1,3-dialkylimidazolium, [(HO₃S)(CH₂)ₙ(NC₅H₆)]⁺, [N(CₙH₂ₙ₊₁)₃(CH₂C₆H₅)]⁺ and [NH(CₙH₂ₙ₊₁)₂((CH₂)ₙOH)]⁺, where m=2, 3 or 4 and n=1, 2 or 3.

The anions of the compound of the formula (I) according to the invention are preferably an anion which is selected from the group consisting of

aryl- and alkylcarboxylates [R⁹C(O)O]⁻ or [R⁹O(CH₂CH₂O)ₙCH₂C(O)O]⁻,
aryl- and alkylsulfonates [R⁹SO₆]⁻,
aryl- and alkylsulfates [R⁹O(CH₂CH₂O)ₙSO₃]⁻, [R⁹OSO₃]⁻ or [HSO₄]⁻, [CF₃SO₃]⁻, [(CF₃SO₂)₂N]⁻, [P(R^{F})_{y}F_{6-y}]⁻, [P(C₆F₅)_{y}F_{6-y}]⁻, [B(CN)₄]⁻ and N(CN)₂⁻,
where R⁹ is a straight-chain or branched alkyl having 1-36 C atoms, preferably 1-20, particularly preferably 10-14 C atoms, or a straight-chain or branched alkenyl having 2-36 C atoms, preferably 2-20, particularly preferably 10-14 C atoms, and one or more double bonds and
R^{F} is a perfluorinated, straight-chain or branched alkyl having 1-36 C atoms, preferably 1-20, particularly preferably 10-14 C atoms, or a perfluorinated, straight-chain or branched alkenyl having 2-36 C atoms, preferably 2-20, particularly preferably 10-14 C atoms, and one or more double bonds, and where
n=2, 3, 4 or 5 and
y=0, 1, 2, 3, 4, 5 or 6.

The compounds according to the invention with carboxylates, in particular ether carboxylates [RO(CH₂CH₂O)ₙCH₂C(O)]⁻, acyl glutamates [RCONHCH(COO⁻)—CH₂CH₂C(O)O]⁻ or sarcosinates [RCON(CH₃)CH₂C(O)O]⁻, particularly preferably stearates or palmitates, alkylsulfates, in particular fatty alcohol ether sulfates [RO(CH₂CH₂O)ₙSO₃]⁻ or fatty alcohol sulfates [ROSO₃]⁻, particularly preferably ethylsulfate, butylsulfate, octylsulfate, 2-ethylhexylsulfate or dodecylsulfate, and alkylsulfonates, in particular sulfosuccinates [RO(CH₂CH₂O)ₙC(O)CH₂CH(COO⁻)—SO₃]⁻, fatty acid isethionates [RO(O)OCH₂CH₂SO₃]⁻ or olefin sulfonates [RCH₂CH═CHCH₂SO₃]⁻ or [RCH₂CH(OH)CH₂CH₂SO₃]⁻, are preferably used in a cosmetic application. By contrast, the compounds according to the invention with the anions [HSO₄]⁻, [CF₃SO₃]⁻, [(CF₃SO₂)₂N]⁻, [P(R^{F})_{y}F_{6-y}]⁻, [P(C₆F₅)_{y}F_{6-y}]⁻, [B(CN)₄]⁻ or [N(CN)₂]⁻ are preferably used as typical ionic liquids.

In a preferred embodiment of the compound according to the invention, the substituent R¹ of the pyrimidinecarboxylic acid ion in a compound of the general formula (I) or (II) is a methyl or ethyl group. The substituents R⁴, R⁵ and R⁶ are very particularly preferably alternatively or simultaneously H.

Very particular preference is given in accordance with the invention to compounds whose general formula is selected from

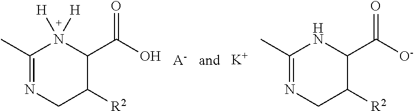

where R² has the meaning H or a hydroxyl group, i.e. very particular preference is given to compounds which contain an ectoin cation, a hydroxyectoin cation, an ectoin anion or a hydroxyectoin anion.

The present invention furthermore relates to a process for the preparation of the compounds according to the invention comprising a cationic component and an anionic component, where a pyrimidinecarboxylic acid derivative represents the cationic or anionic component, in which the neutral pyrimidinecarboxylic acid derivative is quaternised by protonation using a free Brønsted acid or converted into the compound according to the invention by deprotonation by means of a base.

Without restricting generality, Brønsted acids which can be employed in accordance with the invention are, for example, trifluoromethanesulfonic acid, trifluoroacetic acid, HNO₃, H₂SO₄ or HCl.

Without restricting generality, the base employed in accordance with the invention is selected, for example, from the group consisting of a heterocyclic compound, an amine, a tetraalkylammonium hydroxide and a phosphine. The heterocyclic compound here can be, for example, imidazole or a pyridine having an alkyl-SO₃H side chain, where the alkyl side chain has 1-4 C atoms, such as, for example, pyridinopropane-1-sulfonate. The amine used can be, for example, NH₃ or NR₃, where R is an alkyl having 1-4 C atoms. Suitable phosphines are, for example, PR₃, where R is an alkyl having 1-4 C atoms.

The reaction can be carried out at temperatures in the range from 0 to 150° C., preferably at 0 to 50° C. and particularly preferably at room temperature.

The free Brønsted acid or the base is added in this reaction in an amount, based on the neutral pyrimidinecarboxylic acid derivative, which is between a catalytic amount and an equimolar amount of the Brønsted acid or base. The free Brønsted acid or the base is preferably added in an equimolar amount, based on the neutral pyrimidinecarboxylic acid derivative.

Suitable solvents or solvent mixtures are water, alcohols, dialkyl ethers, esters, nitriles, dialkyl carbonates, dichloromethane or mixtures thereof. The solvent is preferably water, methanol, ethanol, i-propanol, acetonitrile, propionitrile, diethyl ether, 1,2-dimethoxyethane, dimethyl carbonate or diethyl carbonate. Water is very particularly preferably used as solvent.

Without restricting generality, further variants of the process according to the invention for the preparation of the compounds according to the invention are described in the working examples.

In addition, other processes which are used for the preparation of classical ionic liquids are also suitable for the preparation of the compounds according to the invention. The person skilled in the art will have no difficulties in falling back on suitable processes here.

The present invention furthermore relates to the use of the compounds according to the invention as ionic liquids.

The compounds according to the invention can be employed as solvent or solvent additive for many synthetic or catalytic reactions, for example Friedel-Crafts acylation and alkylation, Diels-Alder cycloadditions, transition metal- or enzyme-catalysed reactions, hydrogenation and oxidation reactions, Heck reactions, Suzuki couplings, esterifications, isomerisation reactions, hydroformylation reactions, oligomerisation reactions, where the said list is not definitive.

The present invention furthermore relates to the use of the compounds according to the invention as extractant, as heat-transfer medium, as surface-active substance, as plasticiser, as lubricant, as antistatic agent, as flameproofing agent, as non-aqueous electrolyte, optionally in combination with other electrolytes known to the person skilled in the art, or as conductive salt or additive in electrochemical cells.

On use as extractant, the compound according to the invention can be employed for separating off reaction products, but also for separating off impurities, depending on the solubility of the respective component in the compound according to the invention. In addition, the compounds according to the invention can also serve as separating agents in the separation of a plurality of components, for example in the separation of a plurality of components of a mixture by distillation.

In addition, the salts according to the invention can be used as non-aqueous polar substances in suitable reactions, as phase-transfer catalyst, as surfactant (surface-active agent) or as medium for the heterogenisation of homogeneous catalysts.

Further possible applications of the compounds according to the invention are as plasticiser in polymer materials, as flameproofing agent for a number of materials or applications and as conductive salt or additive in various electrochemical cells and applications, for example in galvanic cells, in capacitors or in fuel cells.

The present invention, in particular the compounds indicated as preferred, as described above, furthermore relates to the use of the compounds according to the invention as cosmetic active compound.

The salts according to the invention exhibit advantageous cosmetic actions here, for example antiageing, antiphotoageing, antioxidative actions, melanogenesis-promoting or skin-lightening actions, anticellulite, anti-acne, anticancer, anti-inflammatory action, stabilising action in relation to oxidation-sensitive substances, such as vitamins, perfume components and natural products, stabilising action on photounstable substances, including, for example, UV filters, such as butyl-methoxydibenzoylmethane and ethylhexyl methoxycinnamate, boost actions, for example in relation to the UV protection performance of cosmetic formulations, actions as solubilisers on inadequately soluble components in cosmetic formulations, generally stabilising actions on the formulation properties, such as colour, rheology, odour.

Thus, the compounds according to the invention exhibit skin-protecting and skin-care properties. They can therefore also be used as compatible solutes.

In the original sense, compatible solutes are substances which are involved in the osmoregulation of plants or microorganisms and can be isolated from these organisms.

As compatible solutes, the compounds according to the invention stabilise enzymes, cell structures and other biomolecules in aqueous solutions and organic solvents. They furthermore stabilise, in particular, enzymes against denaturing conditions, such as salts, extreme pH values, surfactants, urea, guanidinium chloride and other compounds.

Of the cosmetic and dermatological applications, the use of the compounds according to the invention for the care of aged, dry or irritated skin should be mentioned in particular. In this case, the compounds according to the invention function primarily as moisturisers for skin and scalp.

The compounds according to the invention can furthermore be employed for the preparation of a composition for the treatment of hair. Introduced into conventional hair-treatment and hair-cleaning compositions, these compounds are capable of restructuring damaged hair and reducing the oxidative damage during oxidative hair colouring. In addition, the salts according to the invention can advantageously be used for the cosmetic treatment of the keratin component, in particular keratin fibres, for example of hair. The compounds according to the invention can be incorporated into hair shampoos, hair rinses, hair cures, permanent-wave and hair-colouring compositions, hair-colouring shampoos, hair tonics, hair-stiffening compositions, hair-setting compositions and/or hair-styling compositions. Application in this respect is preferably carried out during washing and/or during conditioning.

A further area of use of the compounds according to the invention is in the preparation of a cosmetic or dermatological composition for the regeneration and protection and/or revitalisation of the skin by combining the compounds according to the invention with a further active compound, in particular a dried vine shoot extract.

Furthermore, the compounds according to the invention are used for stabilisation of the p53 gene. In this case, these compounds are usually used in the form of a topical composition.

The salts according to the invention can advantageously also be employed in compositions for oral care. In this case, the compounds according to the invention protect the microflora of the skin and mucous membrane, which are important for an intact skin barrier, against stress due to drying out, free radicals, surfactants and high ion concentrations and do not react with cell metabolism.

The compounds according to the invention can furthermore advantageously be used in medicaments and pharmaceutical formulations. In particular, they can be employed for the preparation of a medicament or a dermatological composition for the topical prophylaxis, treatment and/or care of skin diseases, in particular neuro-dermatitis. The medicament or dermatological composition here is preferably mixed together with conventional assistants to give a tincture, lotion, O/W emulsion, W/O emulsion, cream, ointment, hydrogel or spray.

A further area of use of the compounds according to the invention is in the preparation of a medicament for combating diseases caused by the action of airborne dust on the lung tissue and/or cardiovascular diseases associated therewith.

Other pharmaceutical areas of use of the ectoin derivatives are typically in areas in which, for example, trehalose is used as additive. Thus, ectoin derivatives can be used, for example, as protective substance in dried yeast and bacterial cells. Pharmaceutical products, such as non-glycosylated, pharmaceutically active peptides and proteins, for example t-PA, can also be protected using ectoin derivatives.

The present invention furthermore relates to pharmaceutical, cosmetic and dermatological compositions which comprise at least one pyrimidinecarboxylic acid derivative according to the invention. These formulations preferably comprise the compounds according to the invention in amounts of 0.01 to 15% by weight, particularly preferably 0.1 to 10% by weight and very particularly preferably 0.5 to 5% by weight.

The compositions here are usually compositions which can be applied topically, for example cosmetic or dermatological formulations. In this case, the compositions comprise a cosmetically or dermatologically suitable vehicle and, depending on the desired property profile, optionally further suitable ingredients. In the case of pharmaceutical compositions, the compositions comprise a pharmaceutically tolerated excipient and optionally further pharmaceutical active compounds.

For the purposes of the present invention, the term preparation or formulation is also used synonymously alongside the term composition.

All compounds or components which can be used in the compositions are either known and commercially available or can be synthesised by known processes.

The compositions and mixtures described which comprise at least one compound according to the invention may furthermore also comprise pigments, where the layer structure of the pigments is not limited.

The coloured pigment should preferably be skin-coloured or brownish on use of 0.5% to 5% by weight. The choice of a corresponding pigment is familiar to the person skilled in the art.

Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet.

It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-dihydroxyazobenzene | 11920 | orange |
| Solvent Red 3 | 12010 | red |
| 1-(2′-chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| N-(5-Chloro-2,4-dimethoxyphenyl)-4-[[5-[(diethylamino)sulfonyl]- | 12490 | red |
| 2-methoxyphenyl]azo]-3-hydroxynaphthalene-2-carboxamide | | |
| Disperse Yellow 16 | 12700 | yellow |
| 1-(4-Sulfo-1-phenylazo)-4-aminobenzene-5-sulfonic acid | 13015 | yellow |
| 2,4-dihydroxyazobenzene-4′-sulfonic acid | 14270 | orange |
| 2-(2,4-dimethylphenylazo-5-sulfonyl)-1-hydroxynaphthalene-4- | 14700 | red |
| sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonyl-4-chloro-5-carboxy-1-phenylazo)-2-hydroxy- | 15525 | red |
| naphthalene | | |
| 1-(3-Methylphenylazo-4-sulfonyl)-2-hydroxynaphthalene | 15580 | red |
| 1-(4′,(8′)-Sulfonylnaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2-hydroxynaphtha- | 15865 | red |
| lene-3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxypyrazolone- | 19140 | yellow |
| 3-carboxylic acid | | |
| Pigment Yellow 16 | 20040 | yellow |
| 2,6-(4′-Sulfo-2″,4″-dimethyl)bisphenylazo)-1,3-dihydroxybenzene | 20170 | orange |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1-hydroxy- | 27755 | black |
| 7-aminonaphthalene-3,6-disulfonic acid | | |
| 4-[4″-Sulfo-1″-phenylazo)-7′-sulfo-1′-naphthylazo]-1-hydroxy-8- | 28440 | black |
| acetylaminonaphthalene-3,5-disulfonic acid | | |
| Direct Orange 34, 39, 44, 46, 60 | 40215 | orange |
| Food Yellow | 40800 | orange |
| trans-β-Apo-8′-carotene aldehyde (C₃₀) | 40820 | orange |
| trans-Apo-8′-carotinic acid (C₃₀) ethyl ester | 40850 | orange |
| Canthaxanthine | 40850 | orange |
| Acid Blue 1 | 42045 | blue |
| 2,4-Disulfo-5-hydroxy-4′-4″-bis(diethylamino)triphenylcarbinol | 42051 | blue |
| 4-[(4-N-Ethyl-p-sulfobenzylamino)phenyl-(4-hydroxy-2-sulfo- | 42053 | green |
| phenyl)(methylene)-1-(N-ethyl-N-p-sulfobenzyl)-2,5-cyclohexa- | | |
| dienimine] | | |
| Acid Blue 7 | 42080 | blue |
| (N-Ethyl-p-sulfobenzylamino)phenyl-(2-sulfophenyl)methylene- | 42090 | blue |
| (N-ethyl-N-p-sulfobenzyl)-Δ^{2,5}-cyclohexadienimine | | |
| Acid Green 9 | 42100 | green |
| Diethyldisulfobenzyldi-4-amino-2-chlorodi-2-methylfuchsonimmo- | 42170 | green |
| nium | | |
| Basic Violet 14 | 42510 | violet |
| Basic Violet 2 | 42520 | violet |
| 2′-Methyl-4′-(N-ethyl-N-m-sulfobenzyl)amino-4″-(N-diethyl)amino- | 42735 | blue |
| 2-methyl-N-ethyl-N-m-sulfobenzylfuchsonimmonium | | |
| 4′-(N-Dimethyl)amino-4″-(N-phenyl)aminonaphtho-N-dimethyl- | 44045 | blue |
| fuchsonimmonium | | |
| 2-Hydroxy-3,6-disulfo-4,4′-bisdimethylaminonaphthofuchson- | 44090 | green |
| immonium | | |
| Acid Red 52 | 45100 | red |
| 3-(2′-Methylphenylamino)-6-(2′-methyl-4′-sulfophenylamino)-9- | 45190 | violet |
| (2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminium complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinonazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Natural Yellow 6, 19; Natural Red 1 | 75100 | yellow |
| Bixin, Nor-Bixin | 75120 | orange |
| Lycopene | 75125 | yellow |
| trans-alpha-, -beta- or -gamma-Carotene | 75130 | orange |
| Keto and/or hydroxyl derivatives of carotene | 75135 | yellow |
| Guanine or pearlescent agent | 75170 | white |
| 1,7-Bis(4-hydroxy-3-methoxyphenyl)-1,6-heptadiene-3,5-dione | 75300 | yellow |
| Complex salt (Na, Al, Ca) of carminic acid | 75470 | red |
| Chlorophyll a and b; copper compounds of chlorophylls and | 75810 | green |
| chlorophyllines | | |
| Aluminium | 77000 | white |
| Aluminium hydroxide | 77002 | white |
| Water-containing aluminium silicates | 77004 | white |
| Ultramarine | 77007 | blue |
| Pigment Red 101 and 102 | 77015 | red |
| Barium sulfate | 77120 | white |
| Bismuth oxychloride and mixtures thereof with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268 | black |
| | :1 | |
| Chromium oxide | 77288 | green |
| Chromium oxide, water-containing | 77278 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂•7 H₂O | 77745 | red |
| Silver | 77820 | white |
| Titanium dioxide and mixtures thereof with mica | 77891 | white |
| Zinc oxide | 77947 | white |
| 6,7-Dimethyl-9-(1′-D-ribityl)isoalloxazine, lactoflavin | | yellow |
| Sugar dye | | brown |
| Capsanthin, capsorubin | | orange |
| Betanin | | red |
| Benzopyrylium salts, anthocyans | | red |
| Aluminium, zinc, magnesium and calcium stearate | | white |
| Bromothymol Blue | | blue |

Particular preference is given to the types of pearlescent pigment listed below:
1. natural pearlescent pigments, such as, for example,
   1. “pearl essence” (guanine/hypoxanthine mixed crystals from fish scales) and
   2. “mother-of-pearl” (ground mussel shells)
2. monocrystalline pearlescent pigments, such as, for example, bismuth oxychloride (BiOCl)
3. layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under GIN 77163, for example, is particularly advantageous.

Also advantageous are, for example, the following pearlescent pigment types based on mica/metal oxide:

| Group | Coating/layer thickness | Colour |
|---|---|---|
| Silver-white pearlescent pigments | TiO₂: 40-60 nm | silver |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold |
| | | shades |
| | TiO₂/Cr₂O₃ | green |
| | TiO₂/Berlin Blue | dark blue |

Particular preference is given to, for example, the pearlescent pigments available from Merck under the trade names Timiron®, Colorona®, Dichrona®, Xirona® or Ronastar®.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂- and Fe₂O₃-coated SiO₂ particles (“Ronasphere” grades), which are marketed by Merck and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF under the trade name Sicopearl Fantastico.

It may also be advantageous to employ Engelhard/Mearl pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green, blue). The glitter particles here are in the form of mixtures with various assistants and dyes (such as, for example, the dyes with the colour index (CI) numbers 19140, 77007, 77289, 77491).

Particularly suitable pigments in premixes are, for example, Ronastar® Silver or Colorona® Bronze.

The cosmetic composition according to the invention may, in addition, preferably also comprise further active substances, such as, for example, repellents, in particular insect repellents, UV filters, flavone derivatives, chromone derivatives, aryl oximes and parabens.

Most repellent active compounds belong to the substance classes of the amides, alcohols, esters and ethers. Repellents here should usually satisfy the following conditions: they must not evaporate too quickly and must not penetrate into the skin. They must not have a primary irritating or sensitising action on the skin and in addition should be non-toxic. Their efficacy must also be retained when exposed to skin moisture and/or UV radiation.

Preferred repellents are selected from N,N-diethyl-3-methylbenzamide, ethyl 3-(acetylbutylamino)propionate, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, N-(2-ethylhexyl)-8,9,10-trinorborn-5-ene-2,3-dicarboximide, dimethyl carbate, di-n-propyl isocinchomeronate, (R)-p-mentha-1,8-diol, 2-ethylhexane-1,3-diol, N-octylbicyclohepetenedicarboximide, piperonyl butoxide, 1-(2-methylpropyloxycarbonyl)-2-(hydroxyethyl)piperidine (Bayrepel®; Bayer) or mixtures thereof, where they are particularly preferably selected from N,N-diethyl-3-methylbenzamide, ethyl 3-(acetylbutylamino)propionate, 1-(2-methylpropyloxycarbonyl)-2-(hydroxyethyl)piperidine or mixtures thereof.

Parabens are 4-hydroxybenzoic acid esters which are used in free form or as sodium salts for the preservation of compositions in the area of foods, cosmetics and medicaments. The action of the esters is directly proportional to the chain length of the alkyl radical, but conversely the solubility decreases with increasing chain length. As non-dissociating compounds, the esters are substantially pH-independent and act in a pH range of 3.0-8.0. The antimicrobial action mechanism is based on damage of the microbe membranes by the surface activity of the PHB esters and on protein denaturing. In addition, interactions occur with coenzymes. The action is directed against fungi, yeasts and bacteria. The most important parabens as preservatives are methyl 4-hydroxybenzoate, ethyl 4-hydroxybenzoate, propyl 4-hydroxybenzoate and butyl 4-hydroxybenzoate.

Of the aryl oximes, preference is given to the use of 2-hydroxy-5-methyllaurophenone oxime, which is also known as HMLO, LPO or F5. Its suitability for use in cosmetic compositions is disclosed, for example, in DE 41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and other allergic and/or inflammatory diseases of the skin and skin appendages. Compositions according to the invention which, in addition to the said compound(s), additionally comprise an aryl oxime, preferably 2-hydroxy-5-methyllaurophenone oxime, exhibit surprising anti-inflammatory suitability. The compositions here preferably comprise 0.01 to 10% by weight of the aryl oxime, it being particularly preferred for the composition to comprise 0.05 to 5% by weight of aryl oxime.

In accordance with the invention, flavone derivatives are taken to mean flavonoids and coumaranones. In accordance with the invention, flavonoids are taken to mean the glycosides of flavanones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, they are also taken to mean the aglycones, i.e. the sugar-free constituents, and the derivatives of the flavonoids and aglycones. Furthermore, for the purposes of the present invention, the term fiavonoid is also taken to mean anthocyanidine (cyanidine). For the purposes of the present invention, coumaranones are also taken to mean their derivatives. Of the coumaranones, 4,6,3′,4′-tetrahydroxybenzylcoumaranone-3 is preferred.

Chromone derivatives are preferably taken to mean certain chromen-2-one derivatives which are suitable as active compounds for the preventative treatment of human skin and human hair against ageing processes and harmful environmental influences. At the same time, they exhibit a low irritation potential for the skin, have a positive effect on the binding of water in the skin, maintain or increase the elasticity of the skin and thus promote smoothing of the skin. These compounds preferably conform to the following formula:

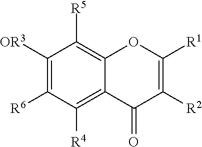

where
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group can be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,
   R³ stands for H or straight-chain or branched C₁- to C₂₀-alkyl groups,
   R⁴ stands for H or OR⁸,
   R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group can be bonded to a primary or secondary carbon atom of the chain and furthermore the alkyl chain may also be interrupted by oxygen, and
   R⁷ stands for H, straight-chain or branched C₁- to C₂₀-alkyl groups, a polyhydroxyl compound, such as, preferably, an ascorbic acid radical or glycosidic radicals, and
   R⁸ stands for H or straight-chain or branched C₁ to C₂₀-alkyl groups, where at least 2 of the substituents R¹, R², R⁴-R⁶ are different from H or at least one substituent from R¹ and R² stands for —C(═O)—R⁷ or —C(═O)—OR⁷.

The proportion of one or more compounds selected from chromone derivatives and coumaranones in a composition is preferably 0.001 to 5% by weight, particularly preferably 0.01 to 2% by weight, based on the entire composition.

The protective action of compositions against oxidative stress or against the action of free radicals can be improved if the compositions comprise one or more antioxidants, where the person skilled in the art is presented with absolutely no difficulties in selecting antioxidants which act suitably quickly or in a delayed manner.

In a preferred embodiment, the composition is therefore a composition for the protection of body cells against oxidative stress, in particular for reducing skin ageing, characterised in that it comprises one or more antioxidants besides the other ingredients.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the general formula A or B

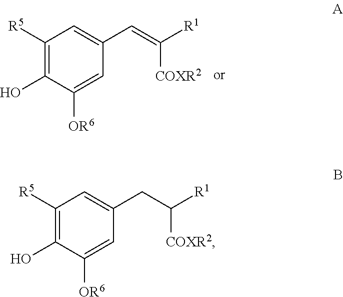

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms, and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Anti-oxidants of this type are usually employed in such compositions with the compounds according to the invention in percent by weight ratios in the range from 1000:1 to 1:1000, preferably in percent by weight ratios of 100:1 to 1:100.

Of the phenols which can be used in accordance with the invention, the polyphenols, some of which are occur as natural products, are particularly interesting for applications in the pharmaceutical, cosmetic or nutrition sector. For example, the flavonoids or bioflavonoids, which are principally known as plant dyes, frequently have an antioxidant potential. Effects of the substitution pattern of mono- and dihydroxyflavones are being investigated by K. Lemanska, H. Szymusiak, B. Tyrakowska, R. Zielinski, I. M. C. M. Rietjens; Current Topics in Biophysics 2000, 24(2), 101-108, where it is observed that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′4′5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881, are investigating the pH dependence of the antioxidant action of hydroxyflavones. Quercetin exhibits the highest activity of the structures investigated over the entire pH range.

Suitable antioxidants are furthermore compounds of the formula (C)

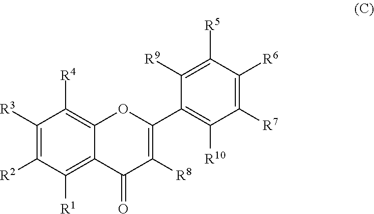

where
R¹ to R¹⁰ may be identical or different and are selected from
H,
OR¹¹,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,
where all OR¹¹, independently of one another, stand for
OH,
straight-chain or branched C₁- to C₂₀-alkoxy groups,
straight-chain or branched C₃- to C₂₀-alkenyloxy groups,
straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
C₃- to C₁₀-cycloalkoxy groups and/or C₃- to C₁₂-cycloalkenyloxy groups, where the rings may each also be bridged by —(CH₂)ₙ— groups, where n=1 to 3, and/or
mono- and/or oligoglycosyl radicals,
   with the proviso that at least 4 radicals from R¹ to R⁷ stand for OH and that at least 2 pairs of adjacent —OH groups are present in the molecule,
or R², R⁵ and R⁶ stand for OH and the radicals R¹, R³, R⁴ and R⁷⁻¹⁰ stand for H,
   as described in German patent application DE-A-102 44 282.

The compositions according to the invention may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In cosmetic applications, vitamins are usually added with the flavonoid-containing premixes or compositions in ranges from 0.01 to 5.0% by weight, based on the total weight. Nutrition-physiclogical applications depend on the respective recommended vitamin need.

Preferred compositions can also serve for sun protection and then also comprise UV filters besides the compounds according to the invention and any other ingredients.

In principle, all UV filters are suitable for combination with the DHA derivatives to be employed in accordance with the invention. Particular preference is given to UV filters whose physiological acceptability has already been demonstrated. Both for UVA and UVB filters, there are many proven substances which are known from the specialist literature, for example

benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-yl-methanesulfonic acid and salts thereof (for example Mexoryl® SX),
2,4,6-trianilino-(p-carbo-2′-ethylhexyl-1′-oxy)-1,3,5-triazine (for example Uvinul® T 150), and
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®, drometrizoles, trisiloxanes, Mexoryl® XL),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl) [and about 6% of methyl[2-[p-[2,2-bis(ethoxycarbonyl]vinyl]phenoxy]-1-methyleneethyl] and about 1.5% of methyl[3-[p-[2,2-bis(ethoxycarbonyl)vinyl)phenoxy)propenyl) and 0.1 to 0.4% of (methylhydrogen)silylene]] (n≈60) (CAS No. 207 574-74-1),
2,2′-methylenebis(6-(2H-benzotriazol-2-yl)-4-(1,1,3,3-tetramethylbutyl)phenol) (CAS No, 103 597-45-1),
2,2′-(1,4-phenylene)bis(1H-benzimidazole-4,6-disulfonic acid, monosodium salt) (CAS No. 180 898-37-7), and
2,4-bis{[4-(2-ethylhexyloxy)-2-hydroxy]phenyl}-6-(4-methoxyphenyl)-1,3,5-triazine (CAS No. 103 597-45-, 187 393-00-6).

Further suitable UV filters are also methoxyflavones corresponding to German patent application DE-A-10232595 or ascorbic acid derivatives in accordance with the PCT application WO 2008/17346 A2.

Organic UV filters are generally incorporated into formulations in an amount of 0.5 to 20 percent by weight, preferably 1-15% by weight.

In order to ensure optimised UV protection, it is furthermore preferred for compositions having light-protection properties also to comprise inorganic UV filters. Conceivable inorganic UV filters are those from the group of the titanium dioxides, such as, for example, coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic compositions in an amount of 0.5 to 20 percent by weight, preferably 2-10% by weight.

Preferred compounds having UV-filtering properties are 3-(4′-methylbenzylidene)dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

The protective action against the harmful effects of UV radiation can be optimised by combining one or more of the said compounds having a UV-filter action.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin are repeatedly discussed in the literature. The encapsulation of the corresponding substances which is proposed here suppresses this effect.
In general, encapsulation of individual UV filters or other ingredients enables formulation problems caused by the interaction of individual composition constituents with one another, such as crystallisation processes, precipitation and agglomerate formation, to be avoided since the interaction is suppressed.

It is therefore preferred for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules particularly preferably to be employed have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions to be employed in accordance with the invention are preferably present in amounts which ensure that the encapsulated UV filters are present in the composition in the percent by weight ratios indicated above.

The compositions to be employed in accordance with the invention may, in addition, comprise further conventional skin-protecting or skin-care active compounds. These can in principle be all active compounds known to the person skilled in the art.

Particularly preferred active compounds, in particular for skin-care compositions, are, for example, also so-called compatible solutes. These are substances which are involved in the osmoregulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and the respective precursors thereof. For the purposes of German patent application DE-A-10133202, osmolytes are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline, and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoin and hydroxyectoin), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacterial cells. Pharmaceutical products, such as non-glycosylated, pharmaceutically active peptides and proteins, for example t-PA, can also be protected with ectoin or its derivatives.

Of the cosmetic applications, particular mention should be made of the use of ectoin and ectoin derivatives for the care of aged, dry or irritated skin. Thus, European patent application EP-A-0 671 161 describes, in particular, that ectoin and hydroxyectoin are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula:

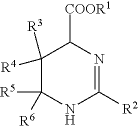

in which R¹ is a radical H or C1-8-alkyl, R² is a radical H or C1-4-alkyl, and R³, R⁴, R⁵ and R⁶ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). In this case, the compositions to be employed in accordance with the invention preferably comprise pyrimidinecarboxylic acids of this type in amounts of up to 15% by weight.

It is particularly preferred here for the compatible solutes to be selected from di-myo-inositol phosphate (DIP), cyclic 2,3-diphosphoglycerate (cDPG), 1,1-diglycerol phosphate (DGP), β-mannosyl glycerate (firoin), β-mannosylglyceramide (firoin-A) and/or dimannosyl diinositol phosphate (DMIP), ectoin, hydroxyectoin or mixtures thereof. In particular, these compatible solutes are located in the water phase, whereas the compounds according to the invention are located in the oil phase on use as compatible solutes.

The compositions according to the invention may furthermore comprise at least one self-tanning agent as further ingredient.

Advantageous self-tanning agents which can be employed are, inter alfa:

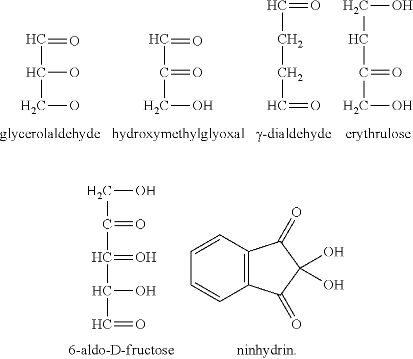

Mention should furthermore be made of 5-hydroxy-1,4-naphthoquinone (juglone), which is extracted from the shells of fresh walnuts

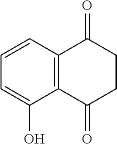

5-hydroxy-1,4-naphthoquinone (juglone)
and 2-hydroxy-1,4-naphthoquinone (lawsone), which occurs in henna leaves,

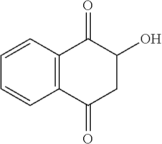

2-hydroxy-1,4-naphthoquinone (lawsone).

Very particular preference is given to 1,3-dihydroxyacetone (DHA), a trifunctional sugar which occurs in the human body, and derivatives thereof

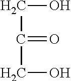

1,3-dihydroxyacetone (DHA).

The compounds according to the invention and any other active compounds can be incorporated into cosmetic, dermatological or pharmaceutical compositions in the usual manner, for example by mixing.

Suitable compositions are those for external use, for example in the form of a cream, lotion, gel or as a solution which can be sprayed onto the skin. Suitable for internal use are administration forms such as capsules, coated tablets, powders, tablet solutions or solutions.

Examples which may be mentioned of application forms of the compositions to be employed are: solutions, suspensions, emulsions, PIT emulsions, pastes, ointments, gels, creams, lotions, powders, soaps, surfactant-containing cleansing preparations, oils, aerosols and sprays. Preferred application forms are also shampoos, tanning lotions and spray products, which are also known from commercial self-tanning studios as spray tans or airbrush tans.

Preferred assistants originate from the group of preservatives, stabilisers, solubilisers, colorants, odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun compositions.

The preferred composition forms also include, in particular, emulsions.

Emulsions are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acids and saturated and/or unsaturated, branched and/or unbranched alcohols having a chain length of 3 to 30 C atoms. Ester oils of this type can then advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of branched and unbranched hydrocarbons and hydrocarbon waxes, silicone oils, dialkyl ethers, or the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may advantageously be selected, for example, from the group of synthetic, semisynthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The aqueous phase of the compositions to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group of silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other fatty substances, as well as water and an emulsifier, as usually used for a formulation of this type.

In a preferred embodiment, the compositions to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix NS 10 (Seppic).

The cosmetic and dermatological compositions may exist in various forms. Thus, they may be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A-43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′ or if isoalkyl derivatives are present, the preferred HLB value of such emulsifiers may also be lower or higher.

It is advantageous to select the fatty alcohol ethoxylates from the group of ethoxylated stearyl alcohols, cetyl alcohols, cetylstearyl alcohols (cetearyl alcohols). Particular preference is given to the following: polyethylene glycol (13) stearyl ether (Steareth-13), polyethylene glycol (14) stearyl ether (Steareth-14), polyethylene glycol (15) stearyl ether (Steareth-15), polyethylene glycol (16) stearyl ether (Steareth-16), polyethylene glycol (17) stearyl ether (Steareth-17), polyethylene glycol (18) stearyl ether (Steareth-18), polyethylene glycol (19) stearyl ether (Steareth-19), polyethylene glycol (20) stearyl ether (Steareth-20), polyethylene glycol (12) isostearyl ether (Isosteareth-12), polyethylene glycol (13) isostearyl ether (Isosteareth-13), polyethylene glycol (14) isostearyl ether (Isosteareth-14), polyethylene glycol (15) isostearyl ether (Isosteareth-15), polyethylene glycol (16) isostearyl ether (Isosteareth-16), polyethylene glycol (17) isostearyl ether (Isosteareth-17), polyethylene glycol (18) isostearyl ether (Isosteareth-18), polyethylene glycol (19) isostearyl ether (Isosteareth-19), polyethylene glycol (20) isostearyl ether (Isosteareth-20), polyethylene glycol (13) cetyl ether (Ceteth-13), polyethylene glycol (14) cetyl ether (Ceteth-14), polyethylene glycol (15) cetyl ether (Ceteth-15), polyethylene glycol (16) cetyl ether (Ceteth-16), polyethylene glycol (17) cetyl ether (Ceteth-17), polyethylene glycol (18) cetyl ether (Ceteth-18), polyethylene glycol (19) cetyl ether (Ceteth-19), polyethylene glycol (20) cetyl ether (Ceteth-20), polyethylene glycol (13) isocetyl ether (Isoceteth-13), polyethylene glycol (14) isocetyl ether (Isoceteth-14), polyethylene glycol (15) isocetyl ether (Isoceteth-15), polyethylene glycol (16) isocetyl ether (Isoceteth-16), polyethylene glycol (17) isocetyl ether (Isoceteth-17), polyethylene glycol (18) isocetyl ether (Isoceteth-18), polyethylene glycol (19) isocetyl ether (Isoceteth-19), polyethylene glycol (20) isocetyl ether (Isoceteth-20), polyethylene glycol (12) oleyl ether (Oleth-12), polyethylene glycol (13) oleyl ether (Oleth-13), polyethylene glycol (14) oleyl ether (Oleth-14), polyethylene glycol (15) oleyl ether (Oleth-15), polyethylene glycol (12) lauryl ether (Laureth-12), polyethylene glycol (12) isolauryl ether (Isolaureth-12), polyethylene glycol (13) cetylstearyl ether (Ceteareth-13), polyethylene glycol (14) cetylstearyl ether (Ceteareth-14), polyethylene glycol (15) cetylstearyl ether (Ceteareth-15), polyethylene glycol (16) cetylstearyl ether (Ceteareth-16), polyethylene glycol (17) cetylstearyl ether (Ceteareth-17), polyethylene glycol (18) cetylstearyl ether (Ceteareth-18), polyethylene glycol (19) cetylstearyl ether (Ceteareth-19), polyethylene glycol (20) cetylstearyl ether (Ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium Laureth-11 carboxylate. An alkyl ether sulfate which can advantageously be used is sodium Laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group of polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group of polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (Steareth-2), glyceryl monolaurate, glyceryl monocaprinate, glyceryl monocaprylate.

Compositions which are preferred in accordance with the invention are also suitable for protecting human skin against ageing processes and against oxidative stress, i.e. against damage caused by free radicals, as are generated, for example, by sunlight, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The composition may comprise cosmetic adjuvants that are usually used in this type of composition, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycol, such as propylene glycol, and/or a polyol, such as glycerol, and oils, waxes and fatty acid esters, such as triglycerides of fatty acids.

The composition may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, use is generally made of the customary propellants, such as alkanes, fluoroalkanes and chlorofluoroalkanes, preferably alkanes.

The compositions to be employed can be prepared with the aid of techniques which are well known to the person skilled in the art.

The compositions, as described above, may comprise, essentially consist of or consist of the said necessary or optional constituents/ingredients.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference.

The examples of the subject-matter according to the invention that are given below serve merely for explanation and in no way restrict the present invention at all. In addition, the invention described can be carried out throughout the entire scope claimed. All compounds or components which can be used in the compositions are either known and commercially available or can be synthesised by known methods. The INCI names of the raw materials used are given (the INCI names are by definition given in English).

The NMR spectra were measured on solutions in deuterated solvents at 20° C. in a Bruker Avance 300 spectrometer with a 5 mm ¹H/BB broadband head with deuterium lock, unless indicated otherwise in the examples. The measurement frequency for the ¹H-NMR is 300.13 MHz.

### EXAMPLES

### Example 1

### 6-Carboxy-2-methyl-1,4,5,6-tetrahydropyrimidinium octanoate

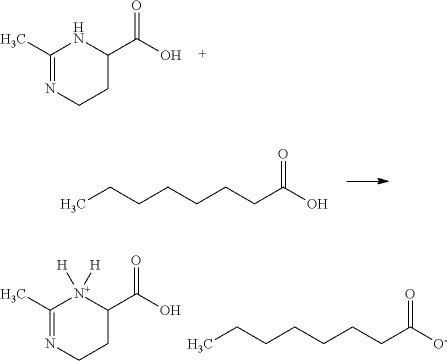

20 g of ectoin are dissolved in 25 ml of water in a 100 ml beaker, and 22.3 ml of octanoic acid are subsequently added at room temperature with stirring. This reaction solution is stirred at room temperature for a further hour and subsequently evaporated to dryness in a rotary evaporator with a water bath at about 60° C., leaving a white, wax-like mass.

### Example 2

### Benzyltrimethylammonium 2-methyl-3,4,5,6-tetrahydropyrimidine-4-carboxylate

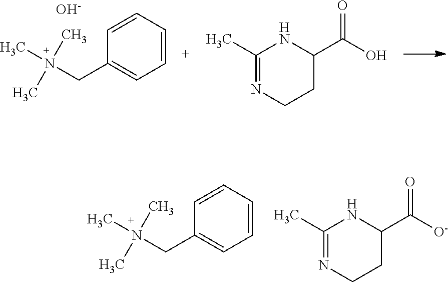

17 g of ectoin are dissolved in 25 ml of water in a 250 ml plastic beaker with magnetic stirrer. 50 g of benzyltrimethylammonium hydroxide are subsequently added at room temperature with stirring. This reaction mixture is stirred at room temperature for about a further half an hour and subsequently evaporated to dryness in a rotary evaporator with a water bath at about 90° C., leaving a clear, slightly viscous liquid.

¹H NMR (d6-DMSO): δ=7.54 (m, 5H), 4.54 (s, 2H), 3.72 (m, 1H), 3.04 (s, 9H), 2.80 (m, 2H), 2.03 (m, 1H), 1.75 (s, 3H), 1.66 (m, 2H).

### Example 3

### 2-Hydroxyethyldimethylammonium 2-methyl-3,4,5,6-tetrahydropyrimidine-4-carboxylate

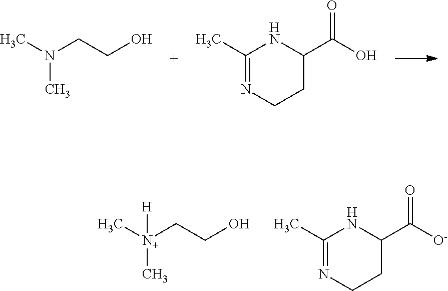

42.65 g of ectoin are dissolved in 60 ml of water in a 250 ml beaker, and 30 ml of 2-(dimethylamino)ethanol are subsequently added at room temperature with stirring. This reaction solution is stirred at room temperature for a further hour and subsequently evaporated to dryness in a rotary evaporator with a water bath at about 60° C., leaving a white solid.

### Example 4

### 1-(3-Sulfopropyl)pyridinium 2-methyl-3,4,5,6-tetrahydropyrimidine-4-carboxylate

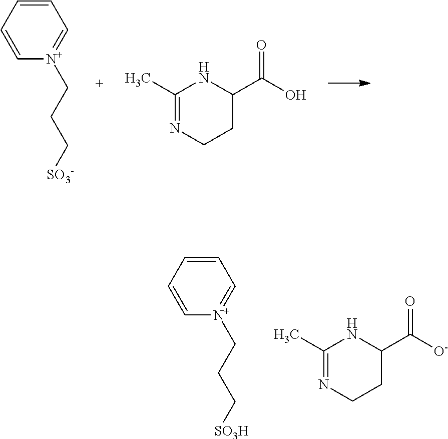

35.26 g of ectoin are dissolved in 50 ml of water in a 250 ml beaker, and 50 g of 3-pyridinopropane-1-sulfonate are subsequently added at room temperature with stirring. This reaction solution is stirred at room temperature for a further hour and subsequently evaporated to dryness in a rotary evaporator with a water bath at about 60° C., leaving a white solid.

### Example 5

### 6-Carboxy-2-methyl-1,4,5,6-tetrahydropyrimidinium hydrogensulfate

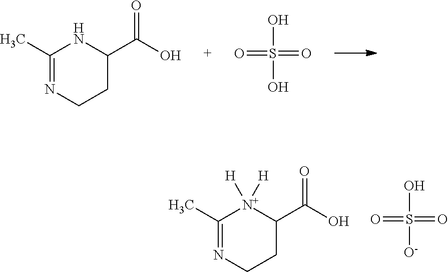

20 g of ectoin are dissolved in 25 ml of water in a 250 ml plastic beaker with magnetic stirrer. 7.83 ml of 96-97% sulfuric acid are subsequently added at room temperature with stirring. The reaction mixture is stirred at room temperature for about a further half an hour and subsequently evaporated to dryness in a rotary evaporator with a water bath at about 90° C., leaving a clear, highly viscous liquid.

### Example 6

### 6-Carboxy-2-methyl-1,4,5,6-tetrahydropyrimidinium trifluoromethanesulfonate

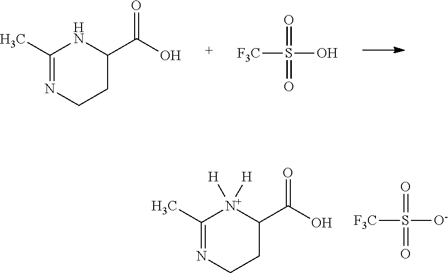

20 g of ectoin are dissolved in 25 ml of water in a 250 ml plastic beaker with magnetic stirrer. 21.16 g of trifluoromethanesulfonic acid are subsequently added at room temperature with stirring. The reaction mixture is stirred at room temperature for about a further half an hour and subsequently evaporated to dryness in a rotary evaporator with a water bath at about 90° C., leaving a clear, slightly viscous liquid.

### Example 7

### 6-Carboxy-2-methyl-1,4,5,6-tetrahydropyrimidinium bis(trifluoromethylsulfonyl)imide

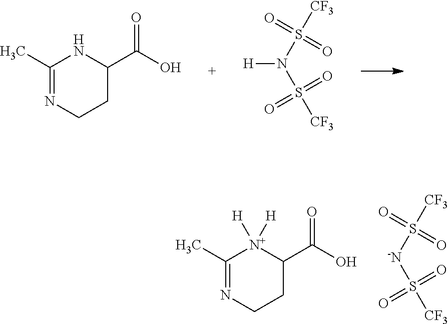

20 g of ectoin are dissolved in 25 ml of water in a 250 ml plastic beaker with magnetic stirrer, 56.63 g of bis(trifluoromethylsulfonyl)imide are subsequently added at room temperature with stirring. The reaction mixture is stirred at room temperature for about a further half an hour and subsequently evaporated to dryness in a rotary evaporator with a water bath at about 90° C., leaving a clear, slightly viscous liquid.

¹H NMR (d6-DMSO): S=4.25 (t, 1H), 3.34 (m, 1H), 3.17 (m, 1H), 2.50 (m, 1H), 2.14 (s, 3H), 2.05 (m, 2H).

### Example 8

### 6-Carboxy-2-methyl-1,4,5,6-tetrahydropyrimidinium palmitate

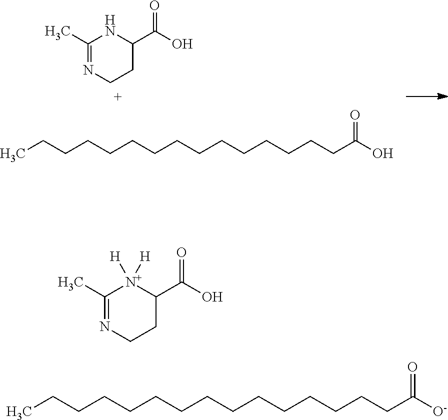

20 g of ectoin are dissolved in 25 ml of water in a 250 ml plastic beaker with magnetic stirrer, and 36.894 g of palmitic acid are subsequently added at room temperature with stirring. This reaction mixture is stirred at room temperature for a further half an hour and subsequently evaporated to dryness in a rotary evaporator with a water bath at about 90° C., leaving a white solid. Melting point 61° C.

### Example 9

### 6-Carboxy-2-methyl-1,4,5,6-tetrahydropyrimidinium stearate

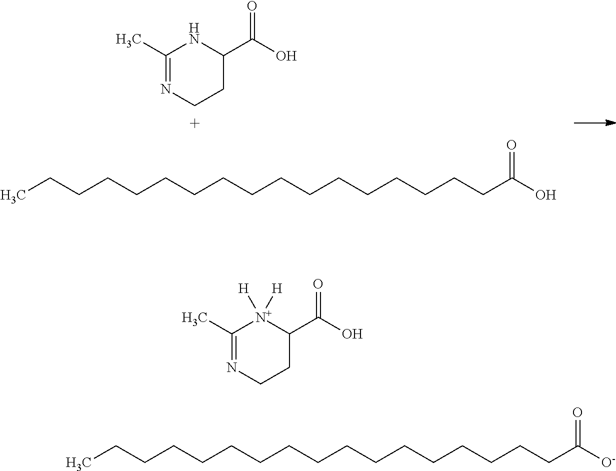

20 g of ectoin are dissolved in 25 ml of water in a 250 ml round-bottomed flask, and 41.352 g of stearic acid are subsequently added at room temperature with stirring. The reaction mixture is stirred at room temperature for a further half an hour and subsequently evaporated to dryness in a rotary evaporator with a water bath at about 90° C., leaving a white solid. Melting point 69° C.

### Example 10

### W/O Emulsion

| | A | B | C | D | E |
|---|---|---|---|---|---|
| Cetyl PEG/PPG-10/1 | 3.00 | 3.00 | 3.00 | 3.00 | 3.00 |
| Dimethicone | | | | | |
| (Abil EM 90) | | | | | |
| Polyglyceryl 4- | 1.50 | 1.50 | 1.50 | 1.50 | 1.50 |
| Isostearate | | | | | |
| (Isolan GI 34) | | | | | |
| Butylphthalimide | 5.00 | 5.00 | 5.00 | 5.00 | 5.00 |
| Isopropylphthalimide | | | | | |
| (Pelemol ® BIP) | | | | | |
| Dimethyl Isosorbide | 5.00 | 5.00 | 5.00 | 5.00 | 5.00 |
| (Arlasolve DMI) | | | | | |
| Benzyltrimethyl- | 1.00 | | | 1.00 | 2.00 |
| ammonium | | | | | |
| 2-methyl-3,4,5,6-tetra- | | | | | |
| hydropyrimidine-4- | | | | | |
| carboxylate | | | | | |
| Uvinul ® A Plus | | 0.84 | 0.84 | 1.00 | |
| (DHHB) | | | | | |
| Ascorbic Acid | | | 0.37 | 1.00 | 3.00 |
| Mineral Oil | 8.00 | 8.00 | 8.00 | 8.00 | 8.00 |
| Ethylhexyl Stearate | 5.00 | 5.00 | 5.00 | 5.00 | 5.00 |
| (Tegosoft ® OS) | | | | | |
| Cyclomethicone (and) | 5.00 | 5.00 | 5.00 | 5.00 | 5.00 |
| Aluminium/Magnesium | | | | | |
| Hydroxide Stearate | | | | | |
| (Gilugel SIL 5) | | | | | |
| Preservative | 1.00 | 1.00 | 1.00 | 1.00 | 1.00 |
| Water | to 100 | to 100 | to 100 | to 100 | to 100 |
| NaCl | 0.50 | 0.50 | 0.50 | 0.50 | 0.50 |
| EDTA | 0.10 | 0.10 | 0.10 | 0.10 | 0.10 |
| Citric Acid q.s. | | | | | |

Preparation: Pelemol BIP, Arlasolve DMI and emulsifiers are initially introduced. Benzyltrimethylammonium 2-methyl-3,4,5,6-tetrahydropyrimidine-4-carboxylate and Uvinul® A Plus are dissolved therein. The remaining constituents of the oil phase are added and mixed homogeneously. The water phase adjusted to pH=4-5 is emulsified in with stirring. The mixture is subsequently homogenised.

### Example 11

### W/O Emulsion

| | A | B | C | D | E |
|---|---|---|---|---|---|
| Cetyl PEG/PPG-10/1 | 3.00 | 3.00 | 3.00 | 3.00 | 3.00 |
| Dimethicone | | | | | |
| (Abil EM 90) | | | | | |
| Polyglyceryl 4- | 1.50 | 1.50 | 1.50 | 1.50 | 1.50 |
| Isostearate | | | | | |
| (Isolan GI 34) | | | | | |
| Butylphthalimide | 5.00 | 5.00 | 5.00 | 5.00 | 5.00 |
| Isopropylphthalimide | | | | | |
| (Pelemol ® BIP) | | | | | |
| Dimethyl Isosorbide | 5.00 | 5.00 | 5.00 | 5.00 | 5.00 |
| (Arlasolve DMI) | | | | | |
| 6-Carboxy-2-methyl- | 1.00 | | | 1.00 | 2.00 |
| 1,4,5,6-tetrahydro- | | | | | |
| pyrimidinium octanoate | | | | | |
| Uvinul ® A Plus | | 0.84 | 0.84 | 1.00 | |
| (DHHB) | | | | | |
| Ascorbic Acid | | | 0.37 | 1.00 | 3.00 |
| Mineral Oil | 8.00 | 8.00 | 8.00 | 8.00 | 8.00 |
| Ethylhexyl Stearate | 5.00 | 5.00 | 5.00 | 5.00 | 5.00 |
| (Tegosoft ® OS) | | | | | |
| Cyclomethicone (and) | 5.00 | 5.00 | 5.00 | 5.00 | 5.00 |
| Aluminium/Magnesium | | | | | |
| Hydroxide Stearate | | | | | |
| (Gilugel SIL 5) | | | | | |
| Preservative | 1.00 | 1.00 | 1.00 | 1.00 | 1.00 |
| Water | to 100 | to 100 | to 100 | to 100 | to 100 |
| NaCl | 0.50 | 0.50 | 0.50 | 0.50 | 0.50 |
| EDTA | 0.10 | 0.10 | 0.10 | 0.10 | 0.10 |
| Citric Acid q.s. | | | | | |

Preparation: Pelemol BIP, Arlasolve DMI and emulsifiers are initially introduced. 6-Carboxy-2-methyl-1,4,5,5-tetrahydropyrimidinium octanoate and Uvinul® A Plus are dissolved therein. The remaining constituents of the oil phase are added and mixed homogeneously. The water phase adjusted to pH=4-5 is emulsified in with stirring. The mixture is subsequently homogenised.

### Example 12

### Water-Resistant Sunscreen Spray

| A | | | |
|---|---|---|---|
| 6-Carboxy-2-methyl-1,4,5,6- | 1.00 | 1.00 | 2.00 |
| tetrahydropyrimidinium | | | |
| hydrogensulfate | | | |
| Diethylhexyl Syringylidenemalonate, | | 0.50 | |
| Caprylic/Capric Triglyceride | | | |
| (Oxynex ® ST Liquid) | | | |
| RonaCare ® AP | | 2.00 | |
| Ascorbyl Palmitate | | | 1.00 |
| Caprylic/Capric Triglyceride | 7.00 | 7.00 | 7.00 |
| (Miglyol 812 N) | | | |
| Butylphthalimide | 10.00 | 10.00 | 10.00 |
| Isopropylphthalimide | | | |
| (Pelemol ® BIP) | | | |
| C12-15 Alkyl Benzoate | 10.00 | 10.00 | 10.00 |
| (Tegosoft ® TN) | | | |
| Phenethyl Benzoate | 5.00 | 5.00 | 5.00 |
| (X-Tend 226) | | | |
| RonaCare ® Tocopherol Acetate | 1.00 | 1.00 | 1.00 |
| B | | | |
| Cyclopentasiloxane | 43.80 | 41.30 | 41.80 |
| (Dow Corning 245) | | | |
| Phenyltrimethicone | 2.00 | 2.00 | 2.00 |
| (Dow Corning 556) | | | |
| Cyclopentasiloxane, Dimethiconol | 20.00 | 20.00 | 20.00 |
| Dow Corning 1501 Fluid | | | |
| Perfume oil (q.s.) | 0.20 | 0.20 | 0.20 |

Preparation: the components of phase A are combined at room temperature and stirred. Phase B is subsequently mixed and added to phase A with stirring.

### Example 13

### Water-Resistant Sunscreen Spray

| A | | | |
|---|---|---|---|
| 2-Hydroxyethyldimethyl- | 1.00 | 1.00 | 2.00 |
| ammonium 2-methyl- | | | |
| 3,4,5,6-tetrahydropyrimidine-4- | | | |
| carboxylate | | | |
| Diethylhexyl Syringylidene- | | 0.50 | |
| malonate, Caprylic/Capric | | | |
| Triglyceride | | | |
| (Oxynex ® ST Liquid) | | | |
| RonaCare ® AP | | 2.00 | |
| Ascorbyl Palmitate | | | 1.00 |
| Caprylic/Capric Triglyceride | 7.00 | 7.00 | 7.00 |
| (Miglyol 812 N) | | | |
| Butylphthalimide | 10.00 | 10.00 | 10.00 |
| Isopropylphthalimide | | | |
| (Pelemol ® BIP) | | | |
| C12-15 Alkyl Benzoate | 10.00 | 10.00 | 10.00 |
| (Tegosoft ® TN) | | | |
| Phenethyl Benzoate | 5.00 | 5.00 | 5.00 |
| (X-Tend 226) | | | |
| RonaCare ® Tocopherol | 1.00 | 1.00 | 1.00 |
| Acetate | | | |
| B | | | |
| Cyclopentasiloxane | 43.80 | 41.30 | 41.80 |
| (Dow Corning 245) | | | |
| Phenyltrimethicone | 2.00 | 2.00 | 2.00 |
| (Dow Corning 556) | | | |
| Cyclopentasiloxane, | 20.00 | 20.00 | 20.00 |
| Dimethiconol | | | |
| Dow Corning 1501 Fluid | | | |
| Perfume oil (q.s.) | 0.20 | 0.20 | 0.20 |

Preparation: the components of phase A are combined at room temperature and stirred. Phase B is subsequently mixed and added to phase A with stirring.

### Example 14

### Pump Hairspray

| | | | |
|---|---|---|---|
| A | | | |
| 6-Carboxy-2-methyl-1,4,5,6- | 1.00 | 2.00 | 4.00 |
| tetrahydropyrimidinium palmitate | | | |
| Ethanol 96% extra pure | to 100 | to 100 | to 100 |
| PVP/VA copolymer | 6.00 | 6.00 | 6.00 |
| PVP/VA W 735 | | | |
| B | | | |
| Diethylhexyl | 0.06 | 0.25 | 0.50 |
| Syringylidenemalonate, | | | |
| Caprylic/Capric Triglyceride | | | |
| (Oxynex ® ST Liquid) | | | |
| PEG-75 Lanolin | 0.20 | 0.20 | 0.20 |
| BHT | | | |
| (Solan E-Low Dioxane) | | | |
| Perfume | 0.10 | 0.10 | 0.10 |
| (Frag 280853 Green Activating) | | | |
| C | | | |
| Water, demineralised | 13.00 | 13.00 | 13.00 |
| Titriplex III | 0.10 | 0.10 | 0.10 |
| PEG-12 Dimethicone | 0.50 | 0.50 | 0.50 |
| Dow Corning 193 Fluid | | | |
| 0.1% D&C Red No 33 (CI 17200) | 0.20 | 0.20 | 0.20 |
| in water | | | |
| PEG-40 Hydrogenated Castor Oil | 1.00 | 1.00 | 1.00 |
| (Cremophor RH 410) | | | |

Preparation: pre-dissolve phase A. Add phase B to phase A with stirring. Pre-mix phase C and add to the remainder, stir until a homogeneous mixture has formed.

### Example 15

### Pump Hairspray

| | | | |
|---|---|---|---|
| A | | | |
| 6-Carboxy-2-methyl-1,4,5,6-tetra- | 1.00 | 2.00 | 4.00 |
| hydropyrimidinium | | | |
| trifluoromethanesulfonate | | | |
| Ethanol 96% extra pure | to 100 | to 100 | to 100 |
| PVP/VA copolymer | 6.00 | 6.00 | 6.00 |
| PVP/VA W 735 | | | |
| B | | | |
| Diethylhexyl | 0.06 | 0.25 | 0.50 |
| Syringylidenemalonate, | | | |
| Caprylic/Capric Triglyceride | | | |
| (Oxynex ® ST Liquid) | | | |
| PEG-75 Lanolin | 0.20 | 0.20 | 0.20 |
| BHT | | | |
| (Solan E-Low Dioxane) | | | |
| Perfume | 0.10 | 0.10 | 0.10 |
| (Frag 280853 Green Activating) | | | |
| C | | | |
| Water, demineralised | 13.00 | 13.00 | 13.00 |
| Titriplex III | 0.10 | 0.10 | 0.10 |
| PEG-12 Dimethicone | 0.50 | 0.50 | 0.50 |
| Dow Corning 193 Fluid | | | |
| 0.1% D&C Red No 33 (CI 17200) | 0.20 | 0.20 | 0.20 |
| in water | | | |
| PEG-40 Hydrogenated Castor Oil | 1.00 | 1.00 | 1.00 |
| (Cremophor RH 410) | | | |

Preparation: pre-dissolve phase A. Add phase B to phase A with stirring. Pre-mix phase C and add to the remainder, stir until a homogeneous mixture has formed.

### Example 16

### W/O Emulsions

| Emulsion | A | B | C | D | E | F |
|---|---|---|---|---|---|---|
| Polyglyceryl 2-Dipolyhydroxy- | 3 | 5 | 3 | | | |
| stearate | | | | | | |
| PEG-30 Dipolyhydroxystearate | | | 2 | 3 | 4 | 5 |
| Sodium Starch | 0.5 | 0.4 | | 0.3 | | 1 |
| Octenylsuccinate | | | | | | |
| Glycine | 0.3 | 0.3 | 0.5 | 0.4 | | |
| Alcohol | | 5 | 2 | 5 | 4 | |
| Magnesium Sulfate | 0.2 | 0.3 | 0.3 | 0.4 | 0.5 | 0.2 |
| C₁₂₋₁₅ Alkyl Benzoate | 5 | 3 | | | 5 | |
| C₁₂₋₁₃ Alkyl Tartrate | | 2 | | | | |
| Butylene Glycol | 5 | | | | 3 | 3 |
| Dicaprylate/Dicaprate | | | | | | |
| Dicaprylyl Ether | | | | | 2 | |
| Mineral Oil | | 4 | | 6 | | 8 |
| Octyldodecanol | 2 | | | | | |
| Dicapryl Caprate | | 2 | | | 2 | 2 |
| Cyclomethicone | 5 | | 5 | 10 | | |
| Dimethicone | | | | 5 | | |
| Isohexadecane | | 1 | | | | |
| Butylene Glycol | 5 | 8 | | | | 3 |
| Propylene Glycol | | | 1 | | 5 | 3 |
| Glycerin | 3 | 5 | 7 | 10 | 3 | 3 |
| C₁₈₋₃₈ acid triglycerides | 0.5 | | 1 | | 1 | |
| Titanium Dioxide | 5 | 6 | 4 | | | 4 |
| Zinc Oxide | 5 | | | | | |
| Bis-Ethylhexyloxyphenol | | 3 | 3 | 2 | | |
| Methoxyphenyltriazine | | | | | | |
| Ethylhexyltriazone | | 4.5 | 3 | | 3 | |
| 1-(3-Sulfopropyl)pyridinium 2- | 2.0 | 0.1 | 1.0 | 0.5 | 3.0 | 1.5 |
| methyl-3,4,5,6-tetrahydro- | | | | | | |
| pyrimidine-4-carboxylate | | | | | | |
| Diethylhexylbutamidotriazone | | | 1.5 | 4 | | |
| Butylmethoxydibenzoylmethane | 2 | 3 | 4 | | 1 | 3 |
| Uvinul ® A Plus | | | | 4 | 2 | |
| Ethylhexyl Methoxycinnamate | | | | | 7 | 5 |
| Taurine | 0.1 | | | 0.5 | 0.2 | |
| Vitamin E Acetate | 0.2 | 0.2 | | 0.3 | 0.1 | 0.5 |
| Na₂H₂EDTA | 0.1 | 0.1 | 0.2 | 0.2 | 0.2 | 0.5 |
| C₈-C₁₆ Alkyl Polyglycoside | 1 | | | | | |
| Perfume, preservatives | q.s. | q.s | q.s | q.s | q.s. | q.s. |
| Dyes, etc. | q.s. | q.s. | q.s. | q.s | q.s. | q.s. |
| Sodium Hydroxide | q.s. | q.s. | q.s. | q.s | q.s. | q.s. |
| Water | to 100.0 | to 100.0 | to 100.0 | to 100.0 | to 100.0 | to 100.0 |

### Example 17

### W/O Emulsions

| Emulsion | A | B | C | D | E | F |
|---|---|---|---|---|---|---|
| Polyglyceryl 2-dipolyhydroxy- | 3 | 5 | 3 | | | |
| stearate | | | | | | |
| PEG-30 Dipolyhydroxystearate | | | 2 | 3 | 4 | 5 |
| Sodium Starch Octenyl- | 0.5 | 0.4 | | 0.3 | | 1 |
| succinate | | | | | | |
| Glycine | 0.3 | 0.3 | 0.5 | 0.4 | | |
| Alcohol | | 5 | 2 | 5 | 4 | |
| Magnesium Sulfate | 0.2 | 0.3 | 0.3 | 0.4 | 0.5 | 0.2 |
| C₁₂₋₁₅ Alkyl Benzoate | 5 | 3 | | | 5 | |
| C₁₂₋₁₃ Alkyl Tartrate | | 2 | | | | |
| Butylene Glycol | 5 | | | | 3 | 3 |
| Dicaprylate/Dicaprate | | | | | | |
| Dicaprylyl Ether | | | | | 2 | |
| Mineral Oil | | 4 | | 6 | | 8 |
| Octyldodecanol | 2 | | | | | |
| Dicapryl Caprate | | 2 | | | 2 | 2 |
| Cyclomethicone | 5 | | 5 | 10 | | |
| Dimethicone | | | | 5 | | |
| Isohexadecane | | 1 | | | | |
| Butylene Glycol | 5 | 8 | | | | 3 |
| Propylene Glycol | | | 1 | | 5 | 3 |
| Glycerin | 3 | 5 | 7 | 10 | 3 | 3 |
| C₁₈₋₃₈ acid triglycerides | 0.5 | | 1 | | 1 | |
| Titanium Dioxide | 5 | 6 | 4 | | | 4 |
| Zinc Oxide | 5 | | | | | |
| Bis-Ethylhexyloxyphenol | | 3 | 3 | 2 | | |
| Methoxyphenyltriazine | | | | | | |
| Ethylhexyltriazone | | 4.5 | 3 | | 3 | |
| 6-Carboxy-2-methyl-1,4,5,6-tetra- | 2.0 | 0.1 | 1.0 | 0.5 | 3.0 | 1.5 |
| hydropyrimidinium stearate | | | | | | |
| Diethylhexylbutamidotriazone | | | 1.5 | 4 | | |
| Butylmethoxydibenzoylmethane | 2 | 3 | 4 | | 1 | 3 |
| Uvinul ® A Plus | | | | 4 | 2 | |
| Ethylhexyl Methoxycinnamate | | | | | 7 | 5 |
| Taurine | 0.1 | | | 0.5 | 0.2 | |
| Vitamin E Acetate | 0.2 | 0.2 | | 0.3 | 0.1 | 0.5 |
| Na₂H₂EDTA | 0.1 | 0.1 | 0.2 | 0.2 | 0.2 | 0.5 |
| C₈-C₁₆ Alkyl Polyglycoside | 1 | | | | | |
| Perfume, preservatives | q.s. | q.s | q.s | q.s | q.s. | q.s. |
| Dyes, etc. | q.s. | q.s. | q.s. | q.s | q.s. | q.s. |
| Sodium Hydroxide | q.s. | q.s. | q.s. | q.s | q.s. | q.s. |
| Water | to 100.0 | to 100.0 | to 100.0 | to 100.0 | to 100.0 | to 100.0 |

### Example 18

### Hair-Care Formulation

Content in g of component per 100 g of formulation

| Component | A | B | C | D | E | F |
|---|---|---|---|---|---|---|
| Disodium EDTA | 0.100 | 0.100 | 0.100 | 0.100 | 0.100 | 0.100 |
| Oxynex ®ST | 2.000 | 2.000 | 2.000 | 2.000 | 2.000 | 2.000 |
| 6-Carboxy-2-methyl-1,4,5,6- | 0.10 | 0.25 | 0.50 | 1.50 | 2.00 | 4.00 |
| tetrahydropyrimidinium | | | | | | |
| bis(trifluoromethylsulfonyl)imide | | | | | | |
| Hexamidine Diisethionate | 0.100 | 0 | 0 | 0 | 0 | 0 |
| Tetrahydrocurcumin | 0 | 0.500 | 0 | 0 | 0 | 0 |
| Glycyrrhetinic Acid | 0 | 0 | 0.300 | 0 | 0 | 0 |
| Thiotaine ®¹ | 0 | 0 | 0 | 5.000 | 0 | 0 |
| N-Undecylenoyl-L-phenylalanine | 0 | 0 | 0 | 0 | 1.000 | 0 |
| N-Acetylglucosamine | 0 | 0 | 0 | 0 | 0 | 2.000 |
| Niacinamide | 5.000 | 5.000 | 5.000 | 5.000 | 5.000 | 5.000 |
| Citric Acid | 0.015 | 0 | 0 | 0 | 0 | 0 |
| Isohexadecane | 3.000 | 3.000 | 3.000 | 3.000 | 3.000 | 3.000 |
| Isopropyl Isostearate | 1.330 | 1.330 | 1.330 | 1.330 | 1.330 | 1.330 |
| Isopropyl N-Laurosylsarcosinate | 0 | 0 | 5.000 | 0 | 0 | 0 |
| Sucrose Polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl Glucoside + Cetearyl | 0.200 | 0.200 | 0.200 | 0.200 | 0.200 | 0.200 |
| Alcohol | | | | | | |
| Behenyl Alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl Alcohol | 0.320 | 0.320 | 0.320 | 0.320 | 0.320 | 0.320 |
| Stearyl Alcohol | 0.480 | 0.480 | 0.480 | 0.480 | 0.480 | 0.480 |
| Tocopheryl Acetate | 0.500 | 0.500 | 0.500 | 0.500 | 0.500 | 0.500 |
| PEG-100 Stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium Dioxide | 0.604 | 0.604 | 0.604 | 0.604 | 0.604 | 0.604 |
| Polyacrylamide + C₁₃₋₁₄ | 3.000 | 2.000 | 2.000 | 2.000 | 2.000 | 2.000 |
| Isoparaffin + Laureth-7 | | | | | | |
| Panthenol | 1.000 | 1.000 | 1.000 | 1.000 | 1.000 | 1.000 |
| Benzyl Alcohol | 0.400 | 0.400 | 0.400 | 0.400 | 0.400 | 0.400 |
| Dimethicone + Dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

### Example 19

### Hair-Care Formulation

Content in g of component per 100 g of formulation

| Component | A | B | C | D | E | F |
|---|---|---|---|---|---|---|
| Disodium EDTA | 0.100 | 0.100 | 0.100 | 0.100 | 0.100 | 0.100 |
| Oxynex ®ST | 2.000 | 2.000 | 2.000 | 2.000 | 2.000 | 2.000 |
| Benzyltrimethylammonium 2- | 0.10 | 0.25 | 0.50 | 1.50 | 2.00 | 4.00 |
| methyl-3,4,5,6-tetrahydro- | | | | | | |
| pyrimidine-4-carboxylate | | | | | | |
| Hexamidine diisethionate | 0.100 | 0 | 0 | 0 | 0 | 0 |
| Tetrahydrocurcumin | 0 | 0.500 | 0 | 0 | 0 | 0 |
| Glycyrrhetinic Acid | 0 | 0 | 0.300 | 0 | 0 | 0 |
| Thiotaine ®¹ | 0 | 0 | 0 | 5.000 | 0 | 0 |
| N-Undecylenoyl-L-Phenylalanine | 0 | 0 | 0 | 0 | 1.000 | 0 |
| N-Acetylglucosamine | 0 | 0 | 0 | 0 | 0 | 2.000 |
| Niacinamide | 5.000 | 5.000 | 5.000 | 5.000 | 5.000 | 5.000 |
| Citric Acid | 0.015 | 0 | 0 | 0 | 0 | 0 |
| Isohexadecane | 3.000 | 3.000 | 3.000 | 3.000 | 3.000 | 3.000 |
| Isopropyl Isostearate | 1.330 | 1.330 | 1.330 | 1.330 | 1.330 | 1.330 |
| Isopropyl N-Laurosylsarcosinate | 0 | 0 | 5.000 | 0 | 0 | 0 |
| Sucrose Polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl Glucoside + Cetearyl | 0.200 | 0.200 | 0.200 | 0.200 | 0.200 | 0.200 |
| Alcohol | | | | | | |
| Behenyl Alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl Alcohol | 0.320 | 0.320 | 0.320 | 0.320 | 0.320 | 0.320 |
| Stearyl Alcohol | 0.480 | 0.480 | 0.480 | 0.480 | 0.480 | 0.480 |
| Tocopheryl Acetate | 0.500 | 0.500 | 0.500 | 0.500 | 0.500 | 0.500 |
| PEG-100 Stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium Dioxide | 0.604 | 0.604 | 0.604 | 0.604 | 0.604 | 0.604 |
| Polyacrylamide + C₁₃₋₁₄ | 3.000 | 2.000 | 2.000 | 2.000 | 2.000 | 2.000 |
| Isoparaffin + Laureth-7 | | | | | | |
| Panthenol | 1.000 | 1.000 | 1.000 | 1.000 | 1.000 | 1.000 |
| Benzyl Alcohol | 0.400 | 0.400 | 0.400 | 0.400 | 0.400 | 0.400 |
| Dimethicone + Dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

### Example 20

### Hair-Care Formulation

Content in g of component per 100 g of formulation

| Component | G | H | I |
|---|---|---|---|
| Disodium EDTA | 0.100 | 0.100 | 0.100 |
| Oxynex ® ST | 2.000 | 2.000 | 2.000 |
| 6-Carboxy-2-methyl-1,4,5,6- | 0.50 | 3.50 | 1.50 |
| tetrahydropyrimidinium octanoate | | | |
| Cetyl Pyridinium Chloride | 0.200 | 0 | 0 |
| Pitera ® | 0 | 10 | 0 |
| Ascorbyl Glycoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl Isostearate | 1.330 | 1.330 | 1.330 |
| Sucrose Polycottonseedate | 0.670 | 0.670 | 0.670 |
| Polymethylsilsesquioxane | 0.250 | 0.250 | 0.250 |
| Cetearyl Glucoside + Cetearyl Alcohol | 0.200 | 0.200 | 0.200 |
| Behenyl Alcohol | 0.400 | 0.400 | 0.400 |
| Ethylparaben | 0.200 | 0.200 | 0.200 |
| Propylparaben | 0.100 | 0.100 | 0.100 |
| Cetyl Alcohol | 0.320 | 0.320 | 0.320 |
| Stearyl Alcohol | 0.480 | 0.480 | 0.480 |
| Tocopheryl Acetate | 0.500 | 0.500 | 0.500 |
| PEG-100 Stearate | 0.100 | 0.100 | 0.100 |
| Glycerin | 7.000 | 7.000 | 7.000 |
| Titanium Dioxide | 0.604 | 0.604 | 0.604 |
| Polyacrylamide + C₁₃₋₁₄ Isoparaffin + | 2.000 | 2.000 | 2.000 |
| Laureth-7 | | | |
| Panthenol | 1.000 | 1.000 | 1.000 |
| Benzyl Alcohol | 0.400 | 0.400 | 0.400 |
| Dimethicone + Dimethiconol | 2.000 | 2.000 | 2.000 |
| Water (to 100 g) | to 100 | to 100 | to 100 |
| TOTAL | 100 | 100 | 100 |

### Example 21

### Hair-Care Formulation

Content in g of component per 100 g of formulation

| Component | G | H | I |
|---|---|---|---|
| Disodium EDTA | 0.100 | 0.100 | 0.100 |
| Oxynex ® ST | 2.000 | 2.000 | 2.000 |
| 6-Carboxy-2-methyl-1,4,5,6- | 0.50 | 3.50 | 1.50 |
| tetrahydropyrimidinium hydrogensulfate | | | |
| Cetyl Pyridinium Chloride | 0.200 | 0 | 0 |
| Pitera ® | 0 | 10 | 0 |
| Ascorbyl Glycoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl Isostearate | 1.330 | 1.330 | 1.330 |
| Sucrose Polycottonseedate | 0.670 | 0.670 | 0.670 |
| Polymethylsilsesquioxane | 0.250 | 0.250 | 0.250 |
| Cetearyl Glucoside + Cetearyl Alcohol | 0.200 | 0.200 | 0.200 |
| Behenyl Alcohol | 0.400 | 0.400 | 0.400 |
| Ethylparaben | 0.200 | 0.200 | 0.200 |
| Propylparaben | 0.100 | 0.100 | 0.100 |
| Cetyl Alcohol | 0.320 | 0.320 | 0.320 |
| Stearyl Alcohol | 0.480 | 0.480 | 0.480 |
| Tocopheryl Acetate | 0.500 | 0.500 | 0.500 |
| PEG-100 Stearate | 0.100 | 0.100 | 0.100 |
| Glycerin | 7.000 | 7.000 | 7.000 |
| Titanium Dioxide | 0.604 | 0.604 | 0.604 |
| Polyacrylamide + C₁₃₋₁₄ Isoparaffin + | 2.000 | 2.000 | 2.000 |
| laureth-7 | | | |
| Panthenol | 1.000 | 1.000 | 1.000 |
| Benzyl Alcohol | 0.400 | 0.400 | 0.400 |
| Dimethicone + Dimethiconol | 2.000 | 2.000 | 2.000 |
| Water (to 100 g) | to 100 | to 100 | to 100 |
| TOTAL | 100 | 100 | 100 |

### Example 22

### O/W Emulsions

| Emulsion | A | B | C | D | E | F |
|---|---|---|---|---|---|---|
| Glyceryl Stearate Citrate | 2.5 | 2 | 3 | | | |
| Sorbitan Stearate | 0.5 | | | 2 | 1.5 | 2 |
| Polyglyceryl-3 Methylglycose | | | | 2.5 | 3 | 3 |
| Distearate | | | | | | |
| Polyglyceryl-2 | | 0.8 | | | | 0.5 |
| Dipolyhydroxystearate | | | | | | |
| Cetearyl Alcohol | | | | 1 | | |
| Stearyl Alcohol | 2 | | | | | 2 |
| Cetyl Alcohol | | 1 | | | 3 | |
| Acrylates/C₁₀₋₃₀ Alkyl | | 0.2 | | | 0.1 | |
| Acrylate Crosspolymer | | | | | | |
| Carbomer | | 0.2 | 0.3 | 0.2 | | |
| Xanthan Gum | 0.4 | | 0.2 | 0.2 | 0.3 | 0.4 |
| C₁₂₋₁₅ Alkyl Benzoate | 5 | 3 | | | 5 | |
| C₁₂₋₁₃ Alkyl Tartrate | | 2 | | | | |
| Butylene Glycol | 5 | | | | 3 | 3 |
| Dicaprylate/Dicaprate | | | | | | |
| Dicaprylyl Ether | | | | | 2 | |
| Octyldodecanol | 2 | | | | | |
| Dicapryl Caprate | | 2 | | | 2 | 2 |
| Cyclomethicone | 5 | | 5 | 10 | | |
| Dimethicone | | | | 5 | | |
| Isohexadecane | | 1 | | | | |
| Butylene Glycol | 5 | 8 | | | | 3 |
| Propylene Glycol | | | 1 | | 5 | 3 |
| Glycerin | 3 | 5 | 7 | 10 | 3 | 3 |
| C₁₈-C₃₈ acid triglycerides | 0.5 | | 1 | | 1 | |
| Titanium Dioxide | 5 | | | 2 | | |
| 2,2′-Methylene bis(6-(2H- | 2.5 | | | | | |
| benzotriazol-2-yl)-(1,1,3,3- | | | | | | |
| tetramethylbutyl)phenol) | | | | | | |
| 2,4,6-Tris-(biphenyl)-1,3,5- | | 2 | | | | |
| triazine | | | | | | |
| Merocyanine coupled to | 6 | | 6 | | 10 | 3 |
| gelatine | | | | | | |
| Benzotriazole coupled to | | 5 | | 10 | | 3 |
| gelatine | | | | | | |
| C₈-C₁₆ Alkylpolyglycoside | 1 | 0.6 | | | | |
| UVASorb ® K2A | | | 2 | | | |
| Uvinul ® A Plus | 2 | | | | | 1 |
| Homosalate | | 5 | | 1 | | |
| Phenylbenzimidazolesulfonic | | | 2 | | | 1 |
| Acid | | | | | | |
| Benzophenone-3 | 2 | | | | 2 | |
| Octyl Salicylate | 5 | 5 | | 2 | | |
| Octocrylene | 2 | | | | 3 | 1 |
| 2-Hydroxyethyldimethyl- | 1.0 | 2.0 | 3.0 | 1.0 | 2.0 | 3.0 |
| ammonium 2-methyl-3,4,5,6- | | | | | | |
| tetrahydropyrimidine-4- | | | | | | |
| carboxylate | | | | | | |
| Bis-Ethylhexyloxyphenol | | 3 | 2 | 1 | | |
| Methoxyphenyltriazine | | | | | | |
| Parsol ® SLX | | | 3 | | | |
| Dihydroxyacetate | | | | | 4 | |
| Taurine | 0.1 | | | 0.5 | 0.2 | |
| 8-Hexadecene-1,16- | | 0.2 | | | | |
| dicarboxylic acid | | | | | | |
| Vitamin E Acetate | 0.2 | 0.2 | | 0.3 | 0.1 | 0.5 |
| Na₂H₂EDTA | 0.1 | 0.1 | 0.2 | 0.2 | 0.2 | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to 100.0 | to 100.0 | to 100.0 | to 100.0 | to 100.0 |

### Example 23

### O/W Emulsions

| Emulsion | A | B | C | D | E | F |
|---|---|---|---|---|---|---|
| Glyceryl Stearate Citrate | 2.5 | 2 | 3 | | | |
| Sorbitan Stearate | 0.5 | | | 2 | 1.5 | 2 |
| Polyglyceryl-3 Methylglycose | | | | 2.5 | 3 | 3 |
| Distearate | | | | | | |
| Polyglyceryl-2 | | 0.8 | | | | 0.5 |
| Dipolyhydroxystearate | | | | | | |
| Cetearyl Alcohol | | | | 1 | | |
| Stearyl Alcohol | 2 | | | | | 2 |
| Cetyl Alcohol | | 1 | | | 3 | |
| Acrylates/C₁₀₋₃₀ Alkyl | | 0.2 | | | 0.1 | |
| Acrylate Crosspolymer | | | | | | |
| Carbomer | | 0.2 | 0.3 | 0.2 | | |
| Xanthan Gum | 0.4 | | 0.2 | 0.2 | 0.3 | 0.4 |
| C₁₂₋₁₅ Alkyl Benzoate | 5 | 3 | | | 5 | |
| C₁₂₋₁₃ Alkyl Tartrate | | 2 | | | | |
| Butylene Glycol | 5 | | | | 3 | 3 |
| Dicaprylate/Dicaprate | | | | | | |
| Dicaprylyl Ether | | | | | 2 | |
| Octyldodecanol | 2 | | | | | |
| Dicapryl Caprate | | 2 | | | 2 | 2 |
| Cyclomethicone | 5 | | 5 | 10 | | |
| Dimethicone | | | | 5 | | |
| Isohexadecane | | 1 | | | | |
| Butylene Glycol | 5 | 8 | | | | 3 |
| Propylene Glycol | | | 1 | | 5 | 3 |
| Glycerin | 3 | 5 | 7 | 10 | 3 | 3 |
| C₁₈-C₃₈ acid triglycerides | 0.5 | | 1 | | 1 | |
| Titanium Dioxide | 5 | | | 2 | | |
| 2,2′-Methylenebis(6-(2H- | 2.5 | | | | | |
| benzotriazol-2-yl)-(1,1,3,3- | | | | | | |
| tetramethylbutyl)phenol) | | | | | | |
| 2,4,6-Tris(biphenyl)-1,3,5- | | 2 | | | | |
| triazine | | | | | | |
| Merocyanine coupled to | 6 | | 6 | | 10 | 3 |
| Gelatine | | | | | | |
| Benzotriazole coupled to | | 5 | | 10 | | 3 |
| Gelatine | | | | | | |
| C₈-C₁₆ Alkyl Polyglycoside | 1 | 0.6 | | | | |
| UVASorb ® K2A | | | 2 | | | |
| Uvinul ® A Plus | 2 | | | | | 1 |
| Homosalate | | 5 | | 1 | | |
| Phenylbenzimidazolesulfonic | | | 2 | | | 1 |
| Acid | | | | | | |
| Benzophenone-3 | 2 | | | | 2 | |
| Octyl Salicylate | 5 | 5 | | 2 | | |
| Octocrylene | 2 | | | | 3 | 1 |
| 6-Carboxy-2-methyl-1,4,5,6- | 1.0 | 2.0 | 3.0 | 1.0 | 2.0 | 3.0 |
| tetrahydropyrimidinium | | | | | | |
| palmitate | | | | | | |
| Bis-Ethylhexyloxyphenol | | 3 | 2 | 1 | | |
| Methoxyphenyltriazine | | | | | | |
| Parsol ® SLX | | | 3 | | | |
| Dihydroxy Acetate | | | | | 4 | |
| Taurine | 0.1 | | | 0.5 | 0.2 | |
| 8-Hexadecene-1,16- | | 0.2 | | | | |
| dicarboxylic Acid | | | | | | |
| Vitamin E Acetate | 0.2 | 0.2 | | 0.3 | 0.1 | 0.5 |
| Na₂H₂EDTA | 0.1 | 0.1 | 0.2 | 0.2 | 0.2 | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium Hydroxide | q.s. | q.s. | q.s. | q.s. | q.s. | q.s. |
| Water | to 100.0 | to 100.0 | to 100.0 | to 100.0 | to 100.0 | to 100.0 |

### Example 24

### O/W Emulsions

| Emulsion | G | H | I | K | L | M |
|---|---|---|---|---|---|---|
| Ceteareth-20 | 1 | 1.5 | 1 | | | |
| Sorbitan Stearate | | | 0.5 | | 0.5 | |
| Glyceryl Stearate SE | | | | 1 | 1 | 1.5 |
| Emulgade F ® | | | | 2.5 | 2.5 | 3 |
| Cetearyl Alcohol | | | | 1 | | |
| Stearyl Alcohol | | | | | 1.5 | |
| Cetyl Alcohol | | | 0.5 | | | 2 |
| Acrylates/C₁₀₋₃₀ Alkyl | 0.2 | 0.4 | 0.3 | 0.1 | | |
| Acrylate Crosspolymer | | | | | | |
| Carbomer | | | | | 0.3 | |
| Xanthan Gum | | | | 0.4 | | 0.4 |
| C₁₂₋₁₅ Alkyl Benzoate | 5 | 3 | | | 5 | |
| 2-Phenyl Benzoate | | 2 | | | | |
| Butylene Glycol | 5 | | | | 3 | 2 |
| Dicaprylate/Dicaprate | | | | | | |
| Dicaprylyl Ether | | | | | 2 | |
| Diethylhexyl Naphthalate | 2 | | | | | |
| Dicapryl Caprate | | 2 | | | 2 | 2 |
| Cyclomethicone | 5 | | 5 | 10 | | |
| Isohexadecane | | | | 5 | | |
| Mineral Oil | | 1 | | | | |
| Propylene Glycol | | | 4 | | | |
| Glycerin | 5 | 7 | 3 | 5 | 6 | 8 |
| C₁₈₋₃₈ acid triglycerides | 0.5 | | 1 | | 1 | |
| Titanium Dioxide | 5 | | 3 | 2 | | |
| NeoHeliopan ® AP | | 2 | | | 1 | 1 |
| Phenylbenzimidazolesulfonic | 1 | | | 1 | 2 | 1 |
| Acid | | | | | | |
| Ethylhexyl | 5 | | 4 | 4 | | |
| Methoxycinnamate | | | | | | |
| Ethylhexyltriazone | | 2 | | 1 | | |
| Diethylhexylbutamido- | 1 | | | | | |
| triazane | | | | | | |
| Butylmethoxydibenzoyl- | 2.5 | | 2 | 2 | | 1 |
| methane | | | | | | |
| Bis-Ethylhexyloxyphenol | 2 | | | | | |
| Methoxyphenyltriazine | | | | | | |
| 4-Methylbenzylidene | 3 | | | | | |
| Camphor | | | | | | |
| Parsol ® SLX | | | | | 2 | |
| 6-Carboxy-2-methyl-1,4,5,6- | 1.0 | 2.0 | 4.0 | 0.5 | 1.5 | 3.0 |
| tetrahydropyrimidinium | | | | | | |
| trifluoromethanesulfonate | | | | | | |
| Creatinine | 0.1 | 0.01 | 0.05 | | | |
| Creatine | 0.5 | 0.2 | 0.1 | | | |
| Liquorice | | | | 0.5 | | |
| Extract/Licochalcone | | | | | | |
| Vitamin E Acetate | 0.2 | | | 0.5 | 0.5 | 0.5 |
| Tapioca Starch | | 3 | | | 2 | |
| Na₂H₂EDTA | 0.1 | | 0.2 | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium Hydroxide | q.s. | q.s. | q.s. | q.s. | q.s. | q.s. |
| Water | to 100.0 | to 100.0 | to 100.0 | to 100.0 | to 100.0 | to 100.0 |

### Example 25

### O/W Emulsions

| Emulsion | G | H | I | K | L | M |
|---|---|---|---|---|---|---|
| Ceteareth-20 | 1 | 1.5 | 1 | | | |
| Sorbitan Stearate | | | 0.5 | | 0.5 | |
| Glyceryl Stearate SE | | | | 1 | 1 | 1.5 |
| Emulgade F ® | | | | 2.5 | 2.5 | 3 |
| Cetearyl Alcohol | | | | 1 | | |
| Stearyl Alcohol | | | | | 1.5 | |
| Cetyl Alcohol | | | 0.5 | | | 2 |
| Acrylates/C₁₀₋₃₀ Alkyl | 0.2 | 0.4 | 0.3 | 0.1 | | |
| Acrylate Crosspolymer | | | | | | |
| Carbomer | | | | | 0.3 | |
| Xanthan Gum | | | | 0.4 | | 0.4 |
| C₁₂₋₁₅ Alkyl Benzoate | 5 | 3 | | | 5 | |
| 2-Phenyl Benzoate | | 2 | | | | |
| Butylene Glycol | 5 | | | | 3 | 2 |
| Dicaprylate/Dicaprate | | | | | | |
| Dicaprylyl Ether | | | | | 2 | |
| Diethylhexyl Naphthalate | 2 | | | | | |
| Dicapryl Caprate | | 2 | | | 2 | 2 |
| Cyclomethicone | 5 | | 5 | 10 | | |
| Isohexadecane | | | | 5 | | |
| Mineral Oil | | 1 | | | | |
| Propylene Glycol | | | 4 | | | |
| Glycerin | 5 | 7 | 3 | 5 | 6 | 8 |
| C₁₈₋₃₈ acid triglycerides | 0.5 | | 1 | | 1 | |
| Titanium Dioxide | 5 | | 3 | 2 | | |
| NeoHeliopan ® AP | | 2 | | | 1 | 1 |
| Phenylbenzimidazolesulfonic | 1 | | | 1 | 2 | 1 |
| Acid | | | | | | |
| Ethylhexyl | 5 | | 4 | 4 | | |
| Methoxycinnamate | | | | | | |
| Ethylhexyltriazone | | 2 | | 1 | | |
| Diethylhexylbutamido- | 1 | | | | | |
| triazane | | | | | | |
| Butylmethoxydibenzoyl- | 2.5 | | 2 | 2 | | 1 |
| methane | | | | | | |
| Bis-Ethylhexyloxyphenol | 2 | | | | | |
| Methoxyphenyltriazine | | | | | | |
| 4-Methylbenzylidene | 3 | | | | | |
| Camphor | | | | | | |
| Parsol ® SLX | | | | | 2 | |
| 1-(3-Sulfopropyl)pyridinium | 1.0 | 2.0 | 4.0 | 0.5 | 1.5 | 3.0 |
| 2-methyl-3,4,5,6-tetrahydro- | | | | | | |
| pyrimidine-4-carboxylate | | | | | | |
| Creatinine | 0.1 | 0.01 | 0.05 | | | |
| Creatine | 0.5 | 0.2 | 0.1 | | | |
| Liquorice Extract/ | | | | 0.5 | | |
| Licochalcone | | | | | | |
| Vitamin E Acetate | 0.2 | | | 0.5 | 0.5 | 0.5 |
| Tapioca Starch | | 3 | | | 2 | |
| Na₂H₂EDTA | 0.1 | | 0.2 | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium Hydroxide | q.s. | q.s. | q.s. | q.s. | q.s. | q.s. |
| Water | to 100.0 | to 100.0 | to 100.0 | to 100.0 | to 100.0 | to 100.0 |

### Example 26

### O/W Emulsions

| Emulsion | N | O | P | Q | R | S |
|---|---|---|---|---|---|---|
| Glyceryl Stearate SE | | 2 | | 2 | | |
| Glyceryl Stearate | 2 | | 2 | | | |
| PEG-40 Stearate | | | 2 | | 1 | |
| PEG-10 Stearate | | | | 2.5 | 1 | |
| Ceteareth-20 | | | | | | 2.6 |
| Sodium Cetyl Phosphate | | | | | 2 | |
| Glyceryl Stearate, | | | | | | 5.4 |
| Ceteareth-12, Ceteareth-20, | | | | | | |
| Cetearyl Alcohol, Cetyl | | | | | | |
| Palmitate | | | | | | |
| Stearic Acid | 3 | 2 | | | 2 | |
| Stearyl Alcohol | | 2 | 2 | | | |
| Stearyl Alcohol | 0.5 | | 2 | | | |
| Cetyl Alcohol | 3 | | | 2 | | |
| Acrylates/C₁₀₋₃₀ Alkyl | | | 0.2 | | 0.4 | |
| Acrylate Crosspolymer | | | | | | |
| Carbomer | | 0.3 | | 0.3 | 0.3 | |
| Xanthan Gum | | 0.3 | 0.4 | | | |
| C₁₂₋₁₅ Alkyl Benzoate | 5 | | | | 5 | 3 |
| 2-Phenyl Benzoate | 5 | | | | | |
| Butylene Glycol | | 5 | | 4 | | 3 |
| Dicaprylate/Dicaprate | | | | | | |
| Dicaprylyl Ether | | 2 | | | 3 | |
| Diethylhexyl Naphthalate | 3 | | | | | |
| Cyclomethicone | 2 | | 10 | 2 | | |
| Isohexadecane | | | | 2 | 3 | |
| Mineral Oil | | | | | 3 | |
| Propanediol | | 3 | | 5 | | |
| Glycerin | 3 | 5 | 10 | 7 | 4 | 5 |
| Titanium Dioxide | 2 | 4 | | | | |
| Zinc Oxide | | | | | 2 | |
| Drometrizole Trisiloxane | | | | | 3 | |
| Ethylhexyl Methoxy- | | 6 | 5 | | | |
| cinnamate | | | | | | |
| Phenylbenzimidazolesulfonic | | 0.5 | 2 | | 1 | |
| Acid | | | | | | |
| Homosalate | 5 | | | 7 | | |
| Butylmethoxydibenzoyl- | | 3 | | | | |
| methane | | | | | | |
| Bis-Ethylhexyloxyphenol | | 2 | 3 | | | |
| Methoxyphenyltriazine | | | | | | |
| Octyl Salicylate | | | | 5 | | |
| Octocrylene | | | | | 3 | |
| 6-Carboxy-2-methyl-1,4,5,6- | 0.25 | 1.5 | 0.5 | 2.5 | 1.0 | 5.0 |
| tetrahydropyrimidinium | | | | | | |
| stearate | | | | | | |
| Parsol ® SLX | 4 | | | | | 5 |
| PVP-Hexadecene | 0.5 | | 1 | | 0.8 | |
| Copolymer | | | | | | |
| Coenzyme Q 10 | 0.2 | 0.02 | | 0.3 | | |
| Vitamin E Acetate | 0.2 | | 0.3 | | 0.8 | 0.5 |
| Na₂H₂EDTA | 0.1 | | | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium Hydroxide | q.s. | q.s. | q.s. | q.s. | q.s. | q.s. |
| Water | to 100.0 | to 100.0 | to 100.0 | to 100.0 | to 100.0 | to 100.0 |

### Example 27

### O/W Emulsions

| Emulsion | N | O | P | Q | R | S |
|---|---|---|---|---|---|---|
| Glyceryl Stearate SE | | 2 | | 2 | | |
| Glyceryl Stearate | 2 | | 2 | | | |
| PEG-40 Stearate | | | 2 | | 1 | |
| PEG-10 Stearate | | | | 2.5 | 1 | |
| Ceteareth-20 | | | | | | 2.6 |
| Sodium Cetyl Phosphate | | | | | 2 | |
| Glyceryl Stearate, | | | | | | 5.4 |
| Ceteareth-12, Ceteareth-20, | | | | | | |
| Cetearyl Alcohol, Cetyl | | | | | | |
| Palmitate | | | | | | |
| Stearic Acid | 3 | 2 | | | 2 | |
| Stearyl Alcohol | | 2 | 2 | | | |
| Stearyl Alcohol | 0.5 | | 2 | | | |
| Cetyl Alcohol | 3 | | | 2 | | |
| Acrylates/C₁₀₋₃₀ Alkyl | | | 0.2 | | 0.4 | |
| Acrylate Crosspolymer | | | | | | |
| Carbomer | | 0.3 | | 0.3 | 0.3 | |
| Xanthan Gum | | 0.3 | 0.4 | | | |
| C₁₂₋₁₅ Alkyl Benzoate | 5 | | | | 5 | 3 |
| 2-Phenyl Benzoate | 5 | | | | | |
| Butylene Glycol | | 5 | | 4 | | 3 |
| Dicaprylate/Dicaprate | | | | | | |
| Dicaprylyl Ether | | 2 | | | 3 | |
| Diethylhexyl Naphthalate | 3 | | | | | |
| Cyclomethicone | 2 | | 10 | 2 | | |
| Isohexadecane | | | | 2 | 3 | |
| Mineral Oil | | | | | 3 | |
| Propanediol | | 3 | | 5 | | |
| Glycerin | 3 | 5 | 10 | 7 | 4 | 5 |
| Titanium Dioxide | 2 | 4 | | | | |
| Zinc Oxide | | | | | 2 | |
| Drometrizole Trisiloxane | | | | | 3 | |
| Ethylhexyl Methoxy- | | 6 | 5 | | | |
| cinnamate | | | | | | |
| Phenylbenzimidazolesulfonic | | 0.5 | 2 | | 1 | |
| Acid | | | | | | |
| Homosalate | 5 | | | 7 | | |
| Butylmethoxydibenzoyl- | | 3 | | | | |
| methane | | | | | | |
| Bis-Ethylhexyloxyphenol | | 2 | 3 | | | |
| Methoxyphenyltriazine | | | | | | |
| Octyl Salicylate | | | | 5 | | |
| Octocrylene | | | | | 3 | |
| 6-Carboxy-2-methyl-1,4,5,6- | 0.25 | 1.5 | 0.5 | 2.5 | 1.0 | 5.0 |
| tetrahydropyrimidinium (bis- | | | | | | |
| trifluoromethylsulfonyl)imide | | | | | | |
| Parsol ® SLX | 4 | | | | | 5 |
| PVP-Hexadecene | 0.5 | | 1 | | 0.8 | |
| Copolymer | | | | | | |
| Coenzyme Q 10 | 0.2 | 0.02 | | 0.3 | | |
| Vitamin E Acetate | 0.2 | | 0.3 | | 0.8 | 0.5 |
| Na₂H₂EDTA | 0.1 | | | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium Hydroxide | q.s. | q.s. | q.s. | q.s. | q.s. | q.s. |
| Water | to 100.0 | to 100.0 | to 100.0 | to 100.0 | to 100.0 | to 100.0 |

### Example 28

### Hydrodispersions (Lotions and Sprays)

| | A | B | C | D | E | F |
|---|---|---|---|---|---|---|
| Glyceryl Stearate Citrate | | 0.40 | | | | |
| Cetyl Alcohol | | | | | 2.00 | |
| Sodium Carbomer | | | | | 0.30 | |
| Acrylates/C₁₀₋₃₀ Alkyl | 0.30 | | 0.30 | 0.40 | 0.10 | 0.10 |
| Acrylate Crosspolymer | | | | | | |
| Ceteareth-20 | | | 1.00 | | | |
| Xanthan Gum | | | | 0.15 | | 0.50 |
| Dimethicone/Vinyl | | | | 5.00 | | 3.00 |
| Dimethicone Crosspolymer | | | | | | |
| UVAsorb ® K2A | | | | | 3.50 | |
| Uvinul ® A Plus | 0.25 | | | 0.50 | 2.00 | 1.50 |
| Butylmethoxydibenzoyl- | 1.20 | | 3.50 | | | |
| methane | | | | | | |
| Bis-Ethylhexyloxyphenol | 2.00 | 2.00 | | 0.25 | | |
| Methoxyphenyltriazine | | | | | | |
| Terephthalidenedicamphor- | | | | | | 0.50 |
| sulfonic Acid | | | | | | |
| Disodium Phenyldibenz- | | | | | | 1.00 |
| imidazoletetrasulfonate | | | | | | |
| Phenylbenzimidazolesulfonic | | | 2.00 | | | |
| Acid | | | | | | |
| Ethylhexyl | 5.00 | | 7.00 | | 5.00 | 8.00 |
| Methoxycinnamate | | | | | | |
| Diethylhexylbutamido- | | | 2.00 | 2.00 | | |
| triazone | | | | | | |
| Ethylhexyltriazone | 4.00 | 3.00 | | | 4.00 | |
| Octocrylene | | | | 10.00 | | 2.50 |
| Benzyltrimethylammonium | 0.25 | 1.5 | 0.5 | 2.5 | 1.0 | 5.0 |
| 2-methyl-3,4,5,6-tetrahydro- | | | | | | |
| pyrimidine-4-carboxylate | | | | | | |
| C₁₂₋₁₅ Alkyl Benzoate | 2.00 | | 2.50 | | | |
| Phenethyl Benzoate | 4.00 | | | 7.50 | | 5.00 |
| C₁₈₋₃₆ Triglyceride Fatty Acid | | | 1.00 | | | |
| Butylene Glycol | | | | | 6.00 | |
| Dicaprylate/Dicaprate | | | | | | |
| Dicaprylyl Carbonate | | 3.00 | | | | |
| Dicaprylyl Ether | | 2.00 | | | | |
| Cyclomethicone | | | | 1.50 | | |
| Lanolin | | | | | 0.35 | |
| PVP-Hexadecene | 0.50 | | 0.50 | | 0.50 | 1.00 |
| Copolymer | | | | | | |
| Ethylhexyloxyglycerin | | 0.75 | | 1.00 | | 0.50 |
| Glycerin | 10.00 | 5.00 | 5.00 | | 5.00 | 15.00 |
| Butylene Glycol | | 7.00 | | | | |
| Glycine Soya | | | | 1.00 | | |
| Vitamin E Acetate | 0.50 | 0.25 | 0 50 | 0.25 | 0.75 | 1.00 |
| α-Glycosylrutin | | | | | 0.25 | |
| Trisodium EDTA | | 1.00 | 1.00 | 0.10 | 0.20 | |
| Iodopropynyl | 0.20 | 0.10 | | | | 0.15 |
| Butylcarbamate | | | | | | |
| Methylparaben | 0.50 | | 0.20 | | 0.15 | |
| Phenoxyethanol | 0.50 | 0.40 | 0.40 | | 1.00 | 0.60 |
| Ethanol | 3.00 | 10.00 | 4.00 | 3.50 | | 1.00 |
| Perfume, dyes | q.s. | q.s. | q.s. | q.s. | q.s. | q.s. |
| Water | to 100 | to 100 | to 100 | to 100 | to 100 | to 100 |
| Neutralisers (sodium | q.s. | q.s. | q.s. | q.s. | q.s. | q.s. |
| hydroxide, potassium | | | | | | |
| hydroxide) | | | | | | |

### Example 29

### Hydrodispersions (Lotions and Sprays)

| | A | B | C | D | E | F |
|---|---|---|---|---|---|---|
| Glyceryl Stearate Citrate | | 0.40 | | | | |
| Cetyl Alcohol | | | | | 2.00 | |
| Sodium Carbomer | | | | | 0.30 | |
| Acrylates/C₁₀₋₃₀ Alkyl | 0.30 | | 0.30 | 0.40 | 0.10 | 0.10 |
| Acrylate Crosspolymer | | | | | | |
| Ceteareth-20 | | | 1.00 | | | |
| Xanthan Gum | | | | 0.15 | | 0.50 |
| Dimethicone/Vinyl | | | | 5.00 | | 3.00 |
| Dimethicone Crosspolymer | | | | | | |
| UVAsorb ® K2A | | | | | 3.50 | |
| Uvinul ® A Plus | 0.25 | | | 0.50 | 2.00 | 1.50 |
| Butylmethoxydibenzoyl- | 1.20 | | 3.50 | | | |
| methane | | | | | | |
| Bis-Ethylhexyloxyphenol | 2.00 | 2.00 | | 0.25 | | |
| Methoxyphenyltriazine | | | | | | |
| Terephthalidenedicamphor- | | | | | | 0.50 |
| sulfonic Acid | | | | | | |
| Disodium Phenyldibenz- | | | | | | 1.00 |
| imidazoletetrasulfonate | | | | | | |
| Phenylbenzimidazolesulfonic | | | 2.00 | | | |
| Acid | | | | | | |
| Ethylhexyl | 5.00 | | 7.00 | | 5.00 | 8.00 |
| Methoxycinnamate | | | | | | |
| Diethylhexylbutamido- | | | 2.00 | 2.00 | | |
| triazone | | | | | | |
| Ethylhexyltriazone | 4.00 | 3.00 | | | 4.00 | |
| Octocrylene | | | | 10.00 | | 2.50 |
| 6-Carboxy-2-methyl-1,4,5,6- | 0.25 | 1.5 | 0.5 | 2.5 | 1.0 | 5.0 |
| tetrahydropyrimidinium | | | | | | |
| octanoate | | | | | | |
| C₁₂₋₁₅ Alkyl Benzoate | 2.00 | | 2.50 | | | |
| Phenethyl Benzoate | 4.00 | | | 7.50 | | 5.00 |
| C₁₈₋₃₆ Triglyceride Fatty Acid | | | 1.00 | | | |
| Butylene Glycol | | | | | 6.00 | |
| Dicaprylate/Dicaprate | | | | | | |
| Dicaprylyl Carbonate | | 3.00 | | | | |
| Dicaprylyl Ether | | 2.00 | | | | |
| Cyclomethicone | | | | 1.50 | | |
| Lanolin | | | | | 0.35 | |
| PVP-Hexadecene | 0.50 | | 0.50 | | 0.50 | 1.00 |
| Copolymer | | | | | | |
| Ethylhexyloxyglycerin | | 0.75 | | 1.00 | | 0.50 |
| Glycerin | 10.00 | 5.00 | 5.00 | | 5.00 | 15.00 |
| Butylene Glycol | | 7.00 | | | | |
| Glycine Soya | | | | 1.00 | | |
| Vitamin E Acetate | 0.50 | 0.25 | 0 50 | 0.25 | 0.75 | 1.00 |
| α-Glycosylrutin | | | | | 0.25 | |
| Trisodium EDTA | | 1.00 | 1.00 | 0.10 | 0.20 | |
| Iodopropynyl | 0.20 | 0.10 | | | | 0.15 |
| Butylcarbamate | | | | | | |
| Methylparaben | 0.50 | | 0.20 | | 0.15 | |
| Phenoxyethanol | 0.50 | 0.40 | 0.40 | | 1.00 | 0.60 |
| Ethanol | 3.00 | 10.00 | 4.00 | 3.50 | | 1.00 |
| Perfume, dyes | q.s. | q.s. | q.s. | q.s. | q.s. | q.s. |
| Water | to 100 | to 100 | to 100 | to 100 | to 100 | to 100 |
| Neutralisers (sodium | q.s. | q.s. | q.s. | q.s. | q.s. | q.s. |
| hydroxide, potassium | | | | | | |
| hydroxide) | | | | | | |

### Example 30

### Aqueous and Aqueous/Alcoholic Formulations

| | A | B | C | D | E | F |
|---|---|---|---|---|---|---|
| Ethanol | 50 | 5 | 2 | 40 | 15 | |
| Hydroxyethylcellulose | 0.5 | | | | | |
| Acrylates/C₁₀₋₃₀ Alkyl Acrylate | | | | 0.3 | 0.6 | |
| Crosspolymer | | | | | | |
| Cocoamidopropylbetaine | | | 0.3 | | | |
| UVASorb ® K2A | | | | | 2 | |
| Uvinul ® A Plus | 5 | | | | | |
| Butylmethoxydibenzoylmethane | 0.5 | | | 3 | | |
| Disodium Phenyldibenz- | | 2 | 1 | | | |
| imidazoletetrasulfonate | | | | | | |
| Phenylbenzimidazolesulfonic | | 5 | 3 | | 2 | 4 |
| Acid | | | | | | |
| Ethylhexyl Methoxycinnamate | 10 | | | | 3 | |
| Diethylhexylbutamidotriazone | | | | 3 | | |
| Ethylhexyltriazone | | | | | 2 | |
| Octocrylene | | | | 5 | | |
| 6-Carboxy-2-methyl-1,4,5,6- | 2.5 | 0.75 | 1.5 | 3.0 | 3.5 | 4.0 |
| tetrahydropyrimidinium | | | | | | |
| hydrogensulfate | | | | | | |
| C₁₂₋₁₅ Alkyl Benzoate | | | | 3 | | |
| C₁₈₋₃₆ Triglyceride Fatty Acid | | | | 1 | | |
| Butylene Glycol | 2 | | | | | |
| Dicaprylate/Dicaprate | | | | | | |
| C₁₂₋₁₃ Alkyl Tartrate | | | | | 5 | |
| Cyclomethicone | 4 | | | 2 | | |
| Insect Repellent ® 3535 | | | | 5 | | |
| Dimethicone | | | | | 3 | |
| PVP-Hexadecene Copolymer | | 0.5 | | 1 | | 0.5 |
| Ethylhexyloxyglycerin | | 0.5 | | | | |
| Glycerin | 5 | 7 | 3 | 8 | | S |
| Butylene Glycol | | | 5 | | 5 | |
| Methylpropanediol | | | | 4 | | |
| Vitamin E Acetate | | 0.3 | 0.2 | 0.5 | | |
| Panthenol | 0.5 | | 0.2 | | | 0.3 |
| Creatinine | | | 0.01 | | 0.02 | |
| Creatine | | | 0.1 | | 0.2 | |
| PEG-40 Hydrogenated Castor | | 0.5 | 0.3 | | | 0.5 |
| Oil | | | | | | |
| Trisodium EDTA | 0.3 | 0.2 | 0.2 | 0.2 | 0.2 | 0.5 |
| Preservatives | q.s. | q.s. | q.s. | q.s. | q.s. | q.s. |
| Sodium Hydroxide | q.s. | q.s. | q.s. | q.s | q.s. | q.s. |
| Perfume, dyes | q.s. | q.s. | q.s. | q.s. | q.s. | q.s. |
| Water | to 100 | to 100 | to 100 | to 100 | to 100 | to 100 |

### Example 31

### Aqueous and Aqueous/Alcoholic Formulations

| | A | E | C | D | E | F |
|---|---|---|---|---|---|---|
| Ethanol | 50 | 5 | 2 | 40 | 15 | |
| Hydroxyethylcellulose | 0.5 | | | | | |
| Acrylates/C₁₀₋₃₀ Alkyl Acrylate | | | | 0.3 | 0.6 | |
| Crosspolymer | | | | | | |
| Cocoamidopropylbetaine | | | 0.3 | | | |
| UVAsorb ® K2A | | | | | 2 | |
| Uvinul ® A Plus | 5 | | | | | |
| Butylmethoxydibenzoylmethane | 0.5 | | | 3 | | |
| Disodium Phenyl- | | 2 | 1 | | | |
| dibenzimidazoletetrasulfonate | | | | | | |
| Phenylbenzimidazolesulfonic | | 5 | 3 | | 2 | 4 |
| Acid | | | | | | |
| Ethylhexyl Methoxycinnamate | 10 | | | | 3 | |
| Diethylhexylbutamidotriazone | | | | 3 | | |
| Ethylhexyltriazone | | | | | 2 | |
| Octocrylene | | | | 5 | | |
| 2-Hydroxyethyldimethyl- | 2.5 | 0.75 | 1.5 | 3.0 | 3.5 | 4.0 |
| ammonium 2-methyl-3,4,5,6- | | | | | | |
| tetrahydropyrimidine-4- | | | | | | |
| carboxylate | | | | | | |
| C₁₂₋₁₅ Alkyl Benzoate | | | | 3 | | |
| C₁₈₋₃₆ Triglyceride Fatty Acid | | | | 1 | | |
| Butylene Glycol | 2 | | | | | |
| Dicaprylate/Dicaprate | | | | | | |
| C₁₂₋₁₃ Alkyl Tartrate | | | | | 5 | |
| Cyclomethicone | 4 | | | 2 | | |
| Insect Repellent ® 3535 | | | | 5 | | |
| Dimethicone | | | | | 3 | |
| PVP-Hexadecene Copolymer | | 0.5 | | 1 | | 0.5 |
| Ethylhexyloxyglycerin | | 0.5 | | | | |
| Glycerin | 5 | 7 | 3 | 8 | | S |
| Butylene Glycol | | | 5 | | 5 | |
| Methylpropanediol | | | | 4 | | |
| Vitamin E Acetate | | 0.3 | 0.2 | 0.5 | | |
| Panthenol | 0.5 | | 0.2 | | | 0.3 |
| Creatinine | | | 0.01 | | 0.02 | |
| Creatine | | | 0.1 | | 0.2 | |
| PEG-40 Hydrogenated Castor | | 0.5 | 0.3 | | | 0.5 |
| Oil | | | | | | |
| Trisodium EDTA | 0.3 | 0.2 | 0.2 | 0.2 | 0.2 | 0.5 |
| Preservatives | q.s. | q.s. | q.s. | q.s. | q.s. | q.s. |
| Sodium Hydroxide | q.s. | q.s. | q.s. | q.s. | q.s. | q.s. |
| Perfume, dyes | q.s. | q.s. | q.s. | q.s. | q.s. | q.s. |
| Water | to 100 | to 100 | to 100 | to 100 | to 100 | to 100 |

### Example 32

### Cosmetic Foams

| Emulsion | A | B | C |
|---|---|---|---|
| Stearic Acid | 2 | 2 | |
| Palmitic Acid | | | 1.5 |
| Cetyl Alcohol | 2.5 | 2 | |
| Stearyl Alcohol | | | 3 |
| PEG-100 Stearate | | | 3.5 |
| PEG-40 Stearate | | 2 | |
| PEG-20 Stearate | 3 | | |
| Sorbitan Stearate | | 0.8 | |
| C₁₂₋₁₅ Alkyl Benzoate | 5 | | |
| C₁₂₋₁₃ Alkyl Tartrate | | | 7 |
| Butylene Glycol | | 6 | |
| Dicaprylate/Dicaprate | | | |
| Dicaprylyl Ether | | | 2 |
| Cyclomethicone | | 2 | 3 |
| Butylene Glycol | 1 | | |
| Isohexadecane | 2 | | |
| Methylpropanediol | | | |
| Propylene glycol | | | 5 |
| Glycerin | 5 | 7 | |
| UVAsorb ® K2A | | | 2 |
| Uvinul A Plus ® | 2 | 3 | |
| 6-Carboxy-2-methyl-1,4,5,6- | 0.5 | 1.0 | 1.5 |
| tetrahydropyrimidinium palmitate | | | |
| Parsol SLX ® | | 3 | |
| Homosalate | | 5 | |
| Phenylbenzimidazolesulfonic Acid | | 2 | 2 |
| Benzophenone-3 | 2 | | |
| Octyl Salicylate | | 5 | |
| Octocrylene | 2 | | |
| Bis-Ethylhexyloxyphenol | | 3 | |
| Methoxyphenyltriazine | | | |
| 2,2′-Methylenebis(6-(2H- | | | 8 |
| benzotriazol-2-yl)-4-(1,1,3,3- | | | |
| tetramethylbutyl)phenol) | | | |
| 2,4,6-Tris(biphenyl)-1,3 5-triazine | 5 | | 4 |
| C₈-C₁₆ Alkyl Polyglycosides | 1 | | |
| Vitamin E Acetate | 0.6 | 0.5 | 0.2 |
| Creatine/Creatinine | | | 0.5 |
| BHT | | | 0.1 |
| Na₂H₂EDTA | 0.50 | | |
| Perfume, preservatives | q.s. | q.s. | q.s. |
| Dyes, etc. | q.s. | q.s. | q.s. |
| Sodium Hydroxide | q.s. | | q.s. |
| Potassium Hydroxide | | q.s. | |
| Water | to 100.0 | to 100.0 | to 100.0 |

### Example 33

### Cosmetic Foams

| Emulsion | A | B | C |
|---|---|---|---|
| Stearic Acid | 2 | 2 | |
| Palmitic Acid | | | 1.5 |
| Cetyl Alcohol | 2.5 | 2 | |
| Stearyl Alcohol | | | 3 |
| PEG-100 Stearate | | | 3.5 |
| PEG-40 Stearate | | 2 | |
| PEG-20 Stearate | 3 | | |
| Sorbitan Stearate | | 0.8 | |
| C₁₂₋₁₅ Alkyl Benzoate | 5 | | |
| C₁₂₋₁₃ Alkyl Tartrate | | | 7 |
| Butylene Glycol | | 6 | |
| Dicaprylate/Dicaprate | | | |
| Dicaprylyl Ether | | | 2 |
| Cyclomethicone | | 2 | 3 |
| Butylene Glycol | 1 | | |
| Isohexadecane | 2 | | |
| Methylpropanediol | | | |
| Propylene Glycol | | | 5 |
| Glycerin | 5 | 7 | |
| UVAsorb ® K2A | | | 2 |
| Uvinul A Plus ® | 2 | 3 | |
| 6-Carboxy-2-methyl-1,4,5,6- | 0.5 | 1.0 | 1.5 |
| tetrahydropyrimidinium trifluoro- | | | |
| methanesulfonate | | | |
| Parsol SLX ® | | 3 | |
| Homosalate | | 5 | |
| Phenylbenzimidazolesulfonic Acid | | 2 | 2 |
| Benzophenone-3 | 2 | | |
| Octyl Salicylate | | 5 | |
| Octocrylene | 2 | | |
| Bis-Ethylhexyloxyphenol | | 3 | |
| Methoxyphenyltriazine | | | |
| 2,2′-Methylenebis(6-(2H- | | | 8 |
| benzotriazol-2-yl)-4-(1,1,3,3- | | | |
| tetramethylbutyl)phenol) | | | |
| 2,4,6-Tris(biphenyl)-1,3 5-triazine | 5 | | 4 |
| C₈-C₁₆ Alkyl Polyglycosides | 1 | | |
| Vitamin E Acetate | 0.6 | 0.5 | 0.2 |
| Creatine/Creatinine | | | 0.5 |
| BHT | | | 0.1 |
| Na₂H₂EDTA | 0.50 | | |
| Perfume, preservatives | q.s. | q.s. | q.s. |
| Dyes, etc. | q.s. | q.s. | q.s. |
| Sodium Hydroxide | q.s. | | q.s. |
| Potassium Hydroxide | | q.s. | |
| Water | to 100.0 | to 100.0 | to 100.0 |

### Example 34

### Cosmetic Foams

| Emulsion | D | E | F | G |
|---|---|---|---|---|
| Stearic Acid | 2 | | | |
| Palmitic Acid | | | 3 | 3 |
| Cetyl Alcohol | 2 | 2 | | |
| Cetylstearyl Alcohol | | | 2 | 2 |
| Stearyl Alcohol | | | | |
| PEG-100 Stearate | | 4 | | |
| PEG-40 Stearate | 2 | | | |
| PEG-20 Stearate | | | 3 | 3 |
| Sorbitan Stearate | 0.8 | | | |
| Tridecyl Trimellitate | | 5 | | |
| C₁₂₋₁₅ Alkyl Benzoate | | | 3 | 3 |
| Butylene Glycol | 8 | | | |
| Dicaprylate/Dicaprate | | | | |
| Octyldodecanol | | 2 | | |
| Cocoglycerides | | | | 2 |
| Dicaprylyl Ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene Glycol | | | | |
| Glycerin | 5 | | 6 | 6 |
| NeoHeliopan ® AP | | 2 | | |
| Phenylbenzimidazole- | 1 | | | 1 |
| sulfonic Acid | | | | |
| 1-(3-Sulfopropyl)pyridinium | 0.25 | 1.5 | 3.0 | 6.0 |
| 2-methyl-3,4,5,6-tetrahydro- | | | | |
| pyrimidine-4-carboxylate | | | | |
| Ethylhexyl Methoxy- | 5 | | 4 | 4 |
| cinnamate | | | | |
| Ethylhexyltriazone | | 2 | | 1 |
| Eusolex T-AVO ® | 2 | | | |
| Diethylhexylbutamido- | 1 | | | |
| triazone | | | | |
| Butylmethoxy- | 2.5 | | 2 | 2 |
| dibenzoylmethane | | | | |
| Bis-Ethylhexyloxyphenol | 2 | | | |
| Methoxyphenyltriazine | | | | |
| Vitamin E Acetate | 0.2 | | 0.3 | 0.3 |
| Na₂H₂EDTA | | | | |
| Perfume, preservatives | | | | |
| Dyes, etc. | | | | |
| Sodium Hydroxide | | q-s. | q.s. | |
| Triethanolamine | q.s. | | | q.s. |
| Water | to 100.0 | to 100.0 | to 100.0 | to 100.0 |

### Example 35

### Cosmetic Foams

| Emulsion | D | E | F | G |
|---|---|---|---|---|
| Stearic Acid | 2 | | | |
| Palmitic Acid | | | 3 | 3 |
| Cetyl Alcohol | 2 | 2 | | |
| Cetylstearyl Alcohol | | | 2 | 2 |
| Stearyl Alcohol | | | | |
| PEG-100 Stearate | | 4 | | |
| PEG-40 Stearate | 2 | | | |
| PEG-20 Stearate | | | 3 | 3 |
| Sorbitan Stearate | 0.8 | | | |
| Tridecyl Trimellitate | | 5 | | |
| C₁₂₋₁₅ Alkyl Benzoate | | | 3 | 3 |
| Butylene Glycol | 8 | | | |
| Dicaprylate/Dicaprate | | | | |
| Octyldodecanol | | 2 | | |
| Cocoglycerides | | | | 2 |
| Dicaprylyl Ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene Glycol | | | | |
| Glycerin | 5 | | 6 | 6 |
| NeoHeliopan ® AP | | 2 | | |
| Phenylbenzimidazole- | 1 | | | 1 |
| sulfonic Acid | | | | |
| 6-Carboxy-2-methyl-1,4,5,6- | 0.25 | 1.5 | 3.0 | 6.0 |
| tetrahydropyrimidinium | | | | |
| stearate | | | | |
| Ethylhexyl Methoxy- | 5 | | 4 | 4 |
| cinnamate | | | | |
| Ethylhexyltriazone | | 2 | | 1 |
| Eusolex T-AVO ® | 2 | | | |
| Diethylhexylbutamido- | 1 | | | |
| triazone | | | | |
| Butylmethoxy- | 2.5 | | 2 | 2 |
| dibenzoylmethane | | | | |
| Bis-Ethylhexyloxyphenol | 2 | | | |
| Methoxyphenyltriazine | | | | |
| Vitamin E Acetate | 0.2 | | 0.3 | 0.3 |
| Na₂H₂EDTA | | | | |
| Perfume, preservatives | | | | |
| Dyes, etc. | | | | |
| Sodium Hydroxide | | q-s. | q.s. | |
| Triethanolamine | q.s. | | | q.s. |
| Water | to 100.0 | to 100.0 | to 100.0 | to 100.0 |

1. Compound comprising a cationic component and an anionic component,
characterised in that a pyrimidinecarboxylic acid derivative represents the cationic component or the anionic component, where ectoin hydrochloride is excluded.

2. Compound according to claim 1,
characterised in that the pyrimidinecarboxylic acid derivative is converted into the cationic component by protonation of a neutral pyrimidinecarboxylic acid derivative or into the anionic component by deprotonation of a neutral pyrimidinecarboxylic acid derivative.

3. Compound according to claim 1,
characterised in that the neutral pyrimidinecarboxylic acid derivative used is ectoin or hydroxyectoin.

4. Compound according to claim 1,
characterised in that it conforms to the general formula (I) 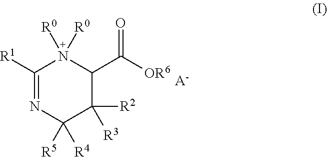
in which the radicals are defined as follows:
R⁰=H or alkyl having 1-12 C atoms,
R¹=H or alkyl having 1-4 C atoms,
R², R³, R⁴, R⁵=each, independently of one another,
H, OH, NH₂ or alkyl having 1-4 C atoms,
R⁶=H or alkyl having 1-8 C atoms,
A⁻=[R⁹C(O)O]⁻, [R^{F}C(O)O]⁻, [R⁹SO₃]⁻, [R^{F}SO₃]⁻, [R⁹OSO₃]⁻, [R^{F}OSO₃]⁻, [(R^{F}SO₂)₂N]⁻, [(R⁹SO₂)₂N]⁻, [(R^{F}C(O))₂N]⁻, [(R⁹C(O))₂N]⁻, [(R^{F}SO₂)(R^{F}C(O))N]⁻, [(R⁹SO₂)(R⁹C(O))N]⁻, [(FSO₂)₃C]⁻, [(R^{F}SO₂)₃C]⁻, [(R⁹SO₂)₃C]⁻, [CCl₃C(O)O]⁻, [(CN)₃C]⁻, [(CN)₂CR⁹]⁻, [(R⁹O(O)C)₂CR⁹]⁻, [P(R^{F})_{y}F_{6-y}]⁻, [P(C₆F₅)_{y}F_{6-y}]⁻, [R⁹₂P(O)O]⁻, [R⁹P(O)O₂]²⁻, [(R⁹O)₂P(O)O]⁻, [(R⁹O)P(O)O₂]²⁻, [(R⁹O)(R⁹)P(O)O]⁻, [R^{F}₂P(O)O]⁻, [R^{F}P(O)O₂]²⁻, [(R^{F})₂P(O)]₂N⁻, [BF_{Z}R^{F}_{4-z}]⁻, [BF_{Z}(CN)_{4-z}]⁻, [B(C₆H₅)₄]⁻, [B(C₆F₅)₄]⁻, [B(OR⁹)₄]⁻, [N(CF₃)₂]⁻, [N(CN)₂]⁻, [AlCl₄]⁻, [SiF₆]²⁻, [R⁹OSO₃]⁻, [HSO₄]⁻, [SO₄]²⁻, [SCN]⁻, [NO₃]⁻, [AlCl₄]⁻, [Al₂Cl₇]⁻, [SnCl₃]⁻, [CO₃]²⁻, [SbF₆]⁻ and [AsF₆]⁻,
where the substituents R^{F} each, independently of one another, denote
perfluorinated and straight-chain or branched alkyl having 1-20 C atoms,
perfluorinated and straight-chain or branched alkenyl having 2-20 C atoms and one or more double bonds,
perfluorinated and saturated, partially or fully unsaturated cycloalkyl having 3-7 C atoms, in particular phenyl, which may be substituted by perfluoroalkyl groups,
where the substituents R^{F} may be bonded to one another in pairs by a single or double bond,
and where one or two carbon atoms of the R^{F} which are not adjacent and are not in the α-position to the heteroatom may be replaced by atoms and/or atom groups selected from the group —O—, —C(O)—, —S—, —S(O)—, —SO₂—, —SO₂O—, —N═, —N═N—, —NH—, —NR′—, —PR′— and —P(O)R′— or may have an end group R′—O—SO₂— or R′—O—C(O)—, where R′ denotes unfluorinated, partially fluorinated or perfluorinated alkyl having 1-6 C atoms, saturated or partially unsaturated cycloalkyl having 3-7 C atoms, unsubstituted or substituted phenyl, including —C₆F₅, or an unsubstituted or substituted heterocycle,
where the substituents R⁹ each, independently of one another, denote
H,
straight-chain or branched alkyl having 1-20 C atoms,
straight-chain or branched alkenyl having 2-20 C atoms and one or more double bonds,
saturated, partially or fully unsaturated cycloalkyl having 3-7 C atoms, in particular phenyl, which may be substituted by alkyl groups,
where a plurality of substituents R⁹ may be bonded to one another in pairs by a single or double bond,
and where one or two carbon atoms of the R⁹ which are not adjacent and are not in the α-position to the heteroatom may be replaced by atoms and/or atom groups selected from the group —O—, —C(O)—, —S—, —S(O)—, —SO₂—, —SO₂O—, —N═, —N═N—, —NH—, —NR′—, —PR′—, —P(O)R′—, —P(O)R′O—, —OP(O)R′O—, —C(O)NH—, —C(O)NR′—, —SO₂NH— and —SO₂NR′, where R′ denotes unfluorinated, partially fluorinated or perfluorinated alkyl having 1-6 C atoms, saturated or partially unsaturated cycloalkyl having 3-7 C atoms, unsubstituted or substituted phenyl, including —C₆F₅, or an unsubstituted or substituted heterocycle,
and where
y=0, 1, 2, 3, 4, 5 or 6 and
z 0, 1, 2, 3 or 4.

5. Compound according to claim 1,
characterised in that it conforms to the general formula (II) 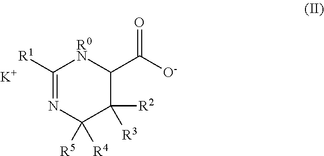
in which the radicals are defined as follows:
R⁰=H or alkyl having 1-12 C atoms,
R¹=H or alkyl having 1-4 C atoms,
R², R³, R⁴, R⁵=each, independently of one another,
H, OH, NH₂ or alkyl having 1-4 C atoms,
K⁺=ammonium [N(R⁷)₄]⁺,
phosphonium [P(R⁷)₄]⁺,
uronium [((R⁷)₂N)—C(═OR⁸)(N(R⁷)₂)]⁺,
thiouronium [((R⁷)₂N)—C(═SR⁸)(N(R)₂)]⁺,
guanidinium [C((N(R⁷)₂)₃]⁺,
sulfonium [S(R⁷)₃]⁺
or a heterocyclic cation [HetN],
where the R⁷, R⁸ each, independently of one another, denote
H, with the proviso that, in the case of [(R⁷)₄N]⁺,
a maximum of two R⁷ are H and that H is excluded for R⁸,
OR′, NR′₂, with the proviso that,
in the case of [(R⁷)₄N]⁺, a maximum of one R⁷ is OR′, NR′₂ and that OR′, NR′₂ are excluded in the case of [((R⁷)₂N)—C(═OR⁸)(N(R⁷)₂)]⁺ and R(R⁷)₂N)—C(═SR⁸)(N(R⁷)₂)⁺,
CN, with the proviso that
CN is excluded in the case of [N(R⁷)₄]⁺, [P(R⁷)₄]⁺, [((R⁷)₂N)—C(═OR⁸)(N(R⁷)₂)]⁺ and [((R⁷)₂N)—C(═SR⁸)(N(R⁷)₂)]⁺,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more R⁷, R⁸ may be partially or fully substituted by halogens, in particular —F and/or —Cl, or partially by —OH, —OR′, —CN, —C(O)OH, —C(O)NR′₂, —SO₂NR′₂, —C(O)X, —SO₂OH, —SO₂X, —NO₂ or —(CH₂)ₙ-phenyl, and where one or two carbon atoms of the R⁷ which are not adjacent and are not in the α-position may be replaced by atoms and/or atom groups selected from the group —O—, —S—, —S(O)—, —SO₂—, —SO₂O—, —C(O)—, —C(O)O—, —N⁺R′₂—, —P(O)R′O—, —C(O)NR′—, —SO₂NR′—, —OP(O)R′O—, —P(O)(NR′₂)NR′—, —PR′₂═N— or —P(O)R′—,
where n=1-4, R′=H, unfluorinated, partially fluorinated or perfluorinated C₁- to C₆-alkyl, C₃- to C₇-cycloalkyl, unsubstituted or substituted phenyl and X=halogen,
and where the heterocyclic cation [HetN]⁺ is selected from the group 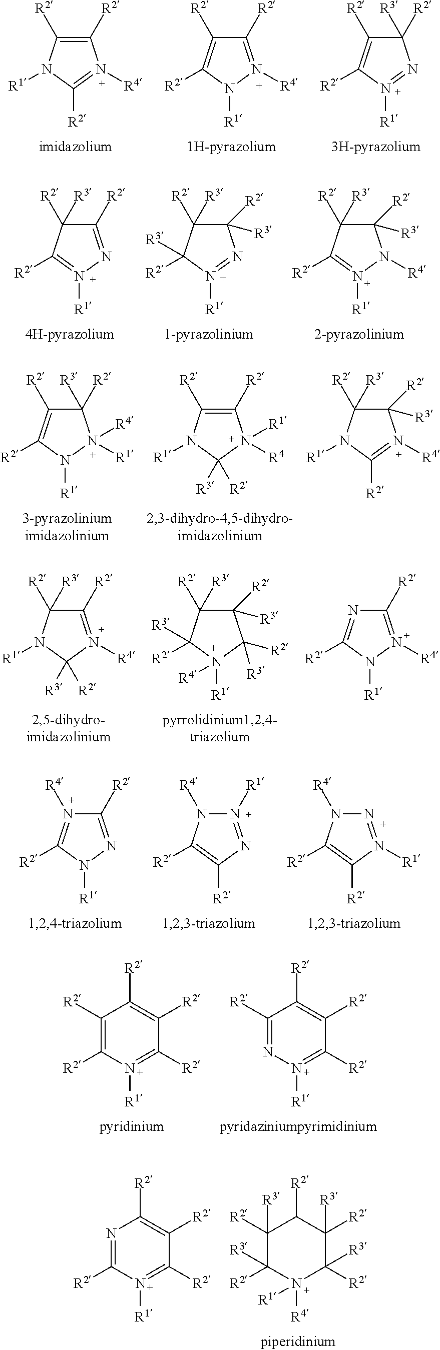 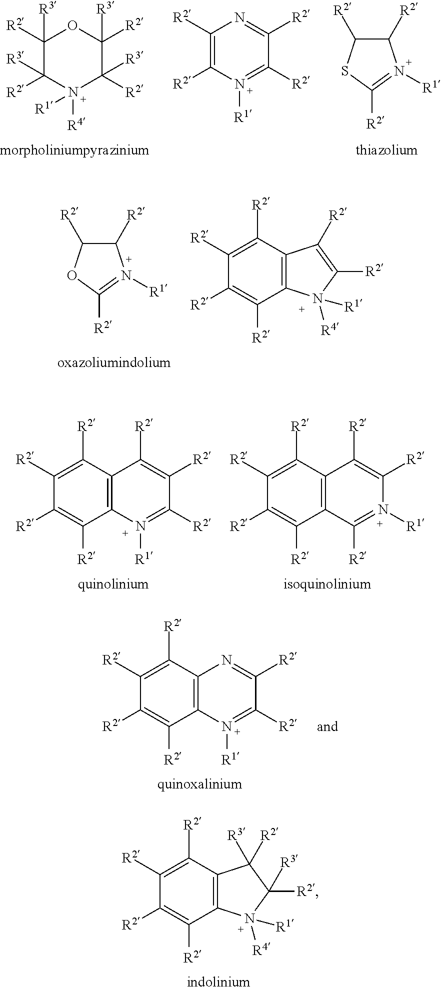
where the substituents R¹′, R²′, R³′ and R⁴′ each, independently of one another, denote
H, —CN, —OR′, —NR′₂, —P(O)R′₂, —P(O)(OR′)₂, —P(O)(NR′₂)₂, —C(O)R′, —C(O)OR′,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
saturated, partially or fully unsaturated heteroaryl,
heteroaryl-C₁-C₆-alkyl or aryl-C₁-C₆-alkyl,
where the substituents R¹′, R²′, R³′ and/or R⁴′ together may also form a ring system,
where one or more substituents R¹′ to R⁴′ may be partially or fully substituted by halogens, in particular —F and/or —Cl, or —OH, —OR′, —CN, —C(O)OH, —C(O)NR′₂, —SO₂NR′₂, —C(O)X, —SO₂OH, —SO₂X, —NO₂ or —(CH₂)ₙ-phenyl, but where R¹′ and R⁴′ cannot simultaneously be fully substituted by halogens,
where one or two substituent R¹′ to R⁴′ carbon atoms which are not adjacent and are not bonded to the heteroatom may be replaced by atoms and/or atom groups selected from the group —O—, —S—, —S(O)—, —SO₂—, —SO₂O—, —C(O)—, —C(O)O—, —N⁺R′₂—, —P(O)R′O—, C(O)NR′—, —SO₂NR′—, —OP(O)R′O—, —P(O)(NR′₂)NR′—, —PR′₂═N— or —P(O)R′—, and where n=1-4, R′=H, unfluorinated, partially fluorinated or perfluorinated C₁- to C₆-alkyl, C₃- to C₇-cycloalkyl, unsubstituted or substituted phenyl and X=halogen.

6. Compound according to claim 4,
characterised in that its general formula is selected from 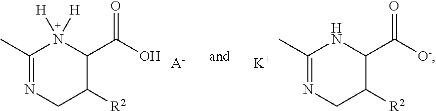
where R² has the meaning H or a hydroxyl group.

7. Compound according to claim 6,
characterised in that A⁻ is selected from the group consisting of [R⁹C(O)O]⁻, [R⁹O(CH₂CH₂O)ₙCH₂C(O)O]⁻, [R⁹SO₃]⁻, [R⁹O(CH₂CH₂O)ₙSO₃]⁻, [R⁹OSO₃]⁻, [HSO₄]⁻, [CF₃SO₃]⁻, [(CF₃SO₂)₂N]⁻, [P(R^{F})_{y}F_{6-y}]⁻, [P(C₆F₅)_{y}F_{6-y}]⁻, [B(CN)₄]⁻ or N(CN)₂⁻,
where R⁹ is a straight-chain or branched alkyl having 1-36 C atoms or a straight-chain or branched alkenyl having 2-36 C atoms and one or more double bonds,
R^{F} is a perfluorinated, straight-chain or branched alkyl having 1-36 C atoms or a perfluorinated, straight-chain or branched alkenyl having 2-36 C atoms and one or more double bonds, and where
n=2, 3, 4 or 5 and
y=0, 1, 2, 3, 4, 5 or 6.

8. Compound according to claim 6,
characterised in that K⁺ is selected from the group consisting of [HetN]⁺ and [N(R⁷)₄]⁺,
where R⁷ in each case, independently of one another, denotes
—H, with the proviso that a maximum of two R⁷ are H,
OR′, NR′₂, with the proviso that a maximum of one R⁷ is OR′, NR′₂,
straight-chain or branched alkyl having 1-20 C atoms,
straight-chain or branched alkenyl having 2-20 C. atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more R⁷ may be partially or fully substituted by halogens, in particular —F and/or —Cl, or partially by —OH, —OR′, —CN, —C(O)OH, —C(O)NR′₂, —SO₂NR′₂, —C(O)X, —SO₂OH, —SO₂X, —NO₂ or —(CH₂)ₙ-phenyl, and where one or two carbon atoms of the R⁷ which are not adjacent and are not in the α-position may be replaced by atoms and/or atom groups selected from the group —O—, —S—, —S(O)—, —SO₂—, —SO₂O—, —C(O)—, —C(O)O—, —N⁺R′₂—, —P(O)R′—, —C(O)NR′—, —SO₂NR′—, —OP(O)R′O—, —P(O)(NR′₂)NR′—, —PR′₂═N— or —P(O)R′—,
where n=1-4, R′=H, unfluorinated, partially fluorinated or perfluorinated C₁- to C₆-alkyl, C₃- to C₇-cycloalkyl, unsubstituted or substituted phenyl and X=halogen,
and where the heterocyclic cation [HetN]⁺ is selected from the group 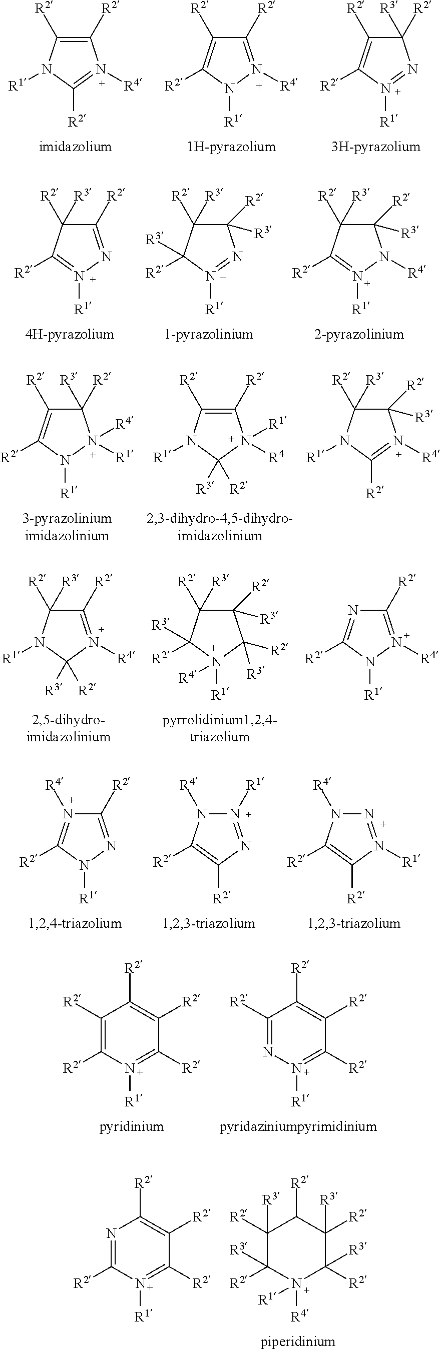 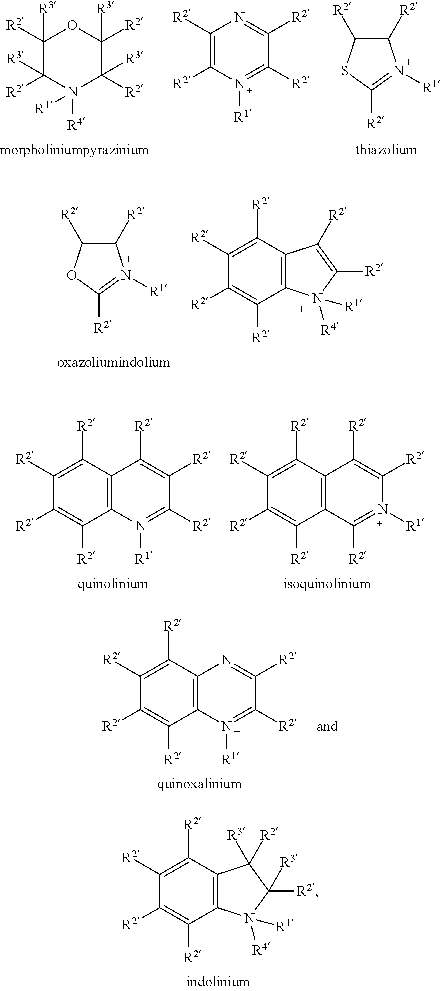
where the substituents R¹′, R²′, R³′ and R⁴′ each, independently of one another, denote
H, —CN, —OR′, —NR′₂, —P(O)R′₂, —P(O)(OR′)₂, —P(O)(NR′₂)₂, —C(O)R′, —C(O)OR′,
straight-chain or branched alkyl having 1-20 C atoms,
straight-chain or branched alkenyl having 2-20 C atoms and one or more double bonds,
straight-chain or branched alkenyl having 2-20 C atoms and one or more triple bonds,
saturated, partially or fully unsaturated cycloalkyl having 3-7 C atoms, which may be substituted by alkyl groups having 1-6 C atoms,
saturated, partially or fully unsaturated heteroaryl,
heteroaryl-C₁-C₆-alkyl or aryl-C₁-C₆-alkyl,
where the substituents R¹′, R²′, R³′ and/or R⁴′ together may also form a ring system,
where one or more substituents R¹′ to R⁴′ may be partially or fully substituted by halogens, in particular —F and/or —Cl, or —OH, —OR′, —CN, —C(O)OH, —C(O)NR′, —SO₂NR′₂, —C(O)X, —SO₂OH, —SO₂X, —NO, or —(CH₂)ₙ-phenyl, but where R¹′ and R⁴′ cannot simultaneously be fully substituted by halogens,
where one or two substituent R¹′ to R⁴′ carbon atoms which are not adjacent and are not bonded to the heteroatom may be replaced by atoms and/or atom groups selected from the group —O—, —S—, —S(O)—, —SO₂—, —SO₂O—, —C(O)—, —C(O)O—, —P(O)R′O—, —C(O)NR′—, —SO₂NR′—, —OP(O)R′O—, —P(O)(NR₂)NR′—, —PR′₂═N— or —P(O)R′—, and where n=1-4, R′=H, unfluorinated partially fluorinated C₁- to C₆-alkyl, C₃- to C₇cycloalkyl, unsubstituted or substituted phenyl and X=halogen.

9. Compound according to claim 8,
characterised in that K⁺ is selected from the group consisting of 1,3-dialkylimidazolium, [(HO₃S)(CH₂)ₙ(NC₅H₅)]⁺, [N(CₙH₂ₙ₊₁)₃(CH₂C₆H₅)]⁺ and [NH(CₙH₂ₙ₊₁)₂((CH₂)ₙOH)]⁺, where m=2, 3 or 4 and n=1, 2 or 3.

10. Process for the preparation of a compound according to claim 1, in which the neutral pyrimidinecarboxylic acid derivative is quaternised by protonation using a free Brønsted acid, preferably selected from the group consisting of trifluoromethanesulfonic acid, trifluoroacetic acid, HNO₃, H₂SO₄ or HCl, or converted into the compound by deprotonation by means of a base, preferably selected from the group consisting of a heterocyclic compound, an amine, a tetraalkylammonium hydroxide and a phosphine.

11. An ionic liquid comprising a compound according to claim 1 as a carrier.

12. (canceled)

13. A pharmaceutical, cosmetic or dermatological composition or food, comprising a compound according to claim 1 and a carrier.

14. (canceled)

15. A process for the preparation of a composition according to claim 13, comprising mixing said compound with a vehicle which is suitable cosmetically or dermatologically or pharmaceutically or for food.
